(19) United States
(12) Patent Application Publication
(10) Pub. No.: US 2025/0020647 A1
(43) Pub. Date: Jan. 16, 2025

(54) METHODS, COMPOSITIONS AND SYSTEMS FOR USING POOLED DONOR SAMPLES FOR LABORATORY TESTING

(71) Applicant: Laboratory Corporation of America Holdings, Burlington, NC (US)

(72) Inventors: Saintedym Wills, Burlington, NC (US); Jessica Chavez, Burlington, NC (US); Ajay Grover, Burlington, NC (US); Andre Valcour, Burlington, NC (US); Russell Phillip Grant, Burlington, NC (US)

(73) Assignee: Laboratory Corporation of America Holdings, Burlington, NC (US)

(21) Appl. No.: 18/772,032

(22) Filed: Jul. 12, 2024

Related U.S. Application Data
(60) Provisional application No. 63/526,364, filed on Jul. 12, 2023.
(60) Provisional application No. 63/579,225, filed on Aug. 28, 2023.

(51) Int. Cl. G01N 33/569

(52) U.S. Cl. CPC G01N 33/56972; G01N 2333/70596; G01N 2800/20

(57) ABSTRACT

Disclosed are methods, compositions and systems for making and using pooled biological samples for laboratory testing. In certain embodiments, the methods and systems may be used for pooling basophil donors for laboratory testing for chronic spontaneous urticaria.

### RELATED APPLICATIONS

This application claims the benefit of U.S. Provisional Application No. 63/526,364 filed on Jul. 12, 2023 and U.S. Provisional Application No. 63/579,225 filed on Aug. 28, 2023. The entire content of said provisional applications is herein incorporated by reference for all purposes.

### FIELD

Disclosed are methods, compositions and systems for making and using pooled biological samples for laboratory testing.

### BACKGROUND

Many diseases require detection of abnormal expression of biological markers (e.g., proteins, cytokines) using flow cytometric or other cellular methods. For example, chronic spontaneous urticaria (CSU) is a prevalent dermatologic disease that can be marked by IgG autoantibodies directed against IgE and IgE receptors on the surface of mast cells or basophils.

Chronic spontaneous urticaria (CSU) is a disorder where urticaria (hives) occurs without an identifiable provoking factor and persists for more than six weeks (Zuberbier et al., The international EAACI/GA²LEN/EuroGuiDerm/APAAACI guideline for the definition, classification, diagnosis, and management of urticaria, Allergy, 2022; 77 (3): 734-766; Kolkhir et al., Autoimmune chronic spontaneous urticaria, J. Allergy Clin. Immunol., 2022, 149 (6): 1819-1831). CSU is thought to result from the pathogenic activation of mast cells and basophils causing them to release histamine and other proinflammatory mediators. Based on experimental and clinical evidence, it has been recognized that a significant proportion of CSU cases have autoimmune etiology (Konstantinou et al., EAACI taskforce position paper: evidence for autoimmune urticaria and proposal for defining diagnostic criteria, Allergy, 2013, 68:27-36; Kolkhir et al., Autoimmune chronic spontaneous urticaria: what we know and what we do not know, J. Allergy Clin. Immunol., 2017; 139:1772-81; Bracken et al., Autoimmune Theories of Chronic Spontaneous Urticaria, Front. Immunol., 2019; 10:627). The autoimmune nature of CSU is further supported by the autologous serum skin test (ASST), an in vivo assay of mast cell activation induced by intradermal injection of a patient's serum into themselves (Sahiner et al., Chronic urticaria: etiology and natural course in children, Int. Arch. Allergy Immunol., 2011, 156:224-30; Hide et al., Autoantibodies against the high-affinity IgE receptor as a cause of histamine release in chronic urticaria, N. Engl. J. Med., 1993; 328 (22): 1599-604; Sabroe et al., 1999, The autologous serum skin test: a screening test for auto-antibodies in chronic idiopathic urticaria, Br. J. Dermatol., 1999, 140:446-52; Konstantinou et al., EAACI/GA (2) LEN task force consensus report: the autologous serum skin test in urticaria, Allergy, 2009, 64:1256-68). Nearly 50% of patients with CSU will develop a wheal-and-flare response at the point of injection when inoculated with their own serum. Unfortunately, positive ASST results are not unique to patients with CSU and have been noted in a substantial proportion of patients with allergic or non-allergic rhinitis, multiple drug allergy syndrome, and even in healthy control subjects (Taskapan et al., Evaluation of autologous serum skin test results in patients with chronic idiopathic urticaria, allergic/non-allergic asthma or rhinitis and healthy people, Clin Exp Dermatol., 2008, 33 (6): 754-8). The utility of the ASST is further limited by the fact that it cannot be used in patients treated with antihistamines, a status that is quite common for patients with CSU in clinical practice (D'Auria et al., Basophil activation test in children with autoimmune chronic spontaneous urticaria: Is it ready for clinical practice? Immunobiology, 2019 January; 224:30-33).

Historically, laboratorians have exploited the tendency of donor basophils to release histamine on exposure to CSU patient serum in the basophil histamine release assay (BHRA) for the in vitro assessment of CSU (Konstantinou et al., 2013, Schoepke et al., Biomarkers and clinical characteristics of autoimmune chronic spontaneous urticaria: Results of the PURIST Study, Allergy, 2019, 74 (12): 2427-2436; Hoffmann et al., The clinical utility of basophil activation testing in diagnosis and monitoring of allergic disease, Allergy, 2015, 70 (11): 1393-405). More recently, flow cytometry has been employed to measure the expression of activation-linked cell surface antigens including CD63 in a more readily automatable indirect basophil activation test (BAT) (Konstantinou et al., 2013; D'Auria et al., 2019; Schoepke et al., 2019; Irinyi et al., Extended diagnostic value of autologous serum skin test and basophil CD63 expression assay in chronic urticaria, Br. J. Dermatol., 2013, 168:656-8; Frezzolini et al., Serum induced basophil CD63 expression by means of a tricolor flow cytometric method for the in vitro diagnosis of chronic urticaria, Allergy, 2006, 61:1071-72006; Szegedi et al., Significant correlation between the CD63 assay and the histamine release assay in chronic urticaria, Br. J. Dermatol., 2006, 155 (1): 67-752006; De Swerdt et al., Detection of basophil-activating IgG autoantibodies in chronic idiopathic urticaria by induction of CD 63, J. Allergy Clin. Immunol., 2005, 116:662-7).

However, the current assay protocol requires three readily available basophil donors who have high activation of anti-FcεRI which can be challenging for both the clinical testing facility and the blood donors. For example, numerous studies have shown that positive indirect BAT results are more frequent in CSU patients than in non-CSU controls (Moñino-Romero et al., Positive Basophil Tests Are Linked to High Disease Activity and Other Features of Autoimmune Chronic Spontaneous Urticaria: A Systematic Review, J. Allergy Clin. Immunol. Pract., 2023: S2213-2198 (23) 00603-7; Marcelino et al., What Basophil Testing Tells Us About CSU Patients-Results of the CORSA Study, Front. Immunol., 2021, 12:742470; HOssein et al., The CD63 basophil activation test as a diagnostic tool for assessing autoimmunity in patients with chronic spontaneous urticaria, Eur. J. Dermatol., 2019, 29 (6): 614-618; Irinyi et al., 2013; Netchiporouk et al., Positive CD63 basophil activation tests are common in children with chronic spontaneous urticaria and linked to high disease activity, Int. Arch. Allergy Immunol., 2016, 171:81-82016; De Swerdt et al., 2005). Furthermore, it has been suggested that positive results for BAT tests might not only be linked to high disease activity in both adults and children but may also serve as predictors for how rapidly the patient will respond to treatment (Netchiporouk et al., 2016; Gericke et al., Serum autoreactivity predicts time to response to omalizumab therapy in chronic spontaneous urticaria, J. Allergy Clin. Immunol., 2017, 139:1059-61; Curto-Barredo et al., Basophil activation test identifies the patients with chronic spontaneous urticaria suffering the most active disease, Immun. Inflamm. Dis., 2016, 4:441-5).

One of the challenges in performing indirect BAT is the intra-individual variability in the response of donor basophils in the assays. While some donor basophils are highly reactive to pathologic serum samples, others are not. Conversely, some donor basophils are non-specifically activated by healthy control serum. In common laboratory practice, this variability in donor responsiveness is accounted for by testing patient samples with multiple donors in separate experiments (Gentinetta et al., 2011). Arbitrary rules are established to assess the results when activation for individual donors varies (Marcelino et al., What Basophil Testing Tells Us About CSU Patients—Results of the CORSA Study, Front. Immunol., 2021, 12:742470; D'Auria et al., 2019).

Thus, there is a need for development of more robust and reliable testing methods.

### SUMMARY OF THE INVENTION

Disclosed are methods and systems to prepare pooled biological samples that may be used for testing subjects for a predetermined indication. In an embodiment, disclosed is a method to pool biological samples for use in testing comprising: screening a plurality of biological samples for a response indicative of the presence of a predetermined indication; and selecting a plurality of the samples that provide the response above a first predetermined threshold to generate the pool. In an embodiment, the method may be used to pool basophil donors from CBC routine testing to provide a consistent and reliable clinically validated assay.

Also disclosed are methods of using pooled samples for testing whether a subject, e.g., a patient, has a predetermined indication of interest. Thus, disclosed is a method to test for a response indicative of the presence of a predetermined indication comprising in a sample from a subject comprising the steps of: generating a plurality of pooled samples that provide the response above a first predetermined threshold and optionally, do not exhibit a non-specific response; and adding a portion of the sample from the subject to the pooled samples to determine if the sample generates the response.

Also disclosed are compositions, kits, systems and computer program products for performing the disclosed methods or any of the steps of the disclosed methods.

### BRIEF DESCRIPTION OF THE FIGURES

The patent or application file contains at least one drawing executed in color. Copies of this patent or patent application publication with color drawing(s) will be provided by the Office upon request and payment of the necessary fee.

The disclosure may be better understood by reference to the following non-limiting figures.

FIG. 1 shows a method for generating and using donor pools for patient testing in accordance with one embodiment of the disclosure.

FIG. 2 shows a system for generating and using donor pools for patient testing in accordance with an embodiment of the disclosure.

FIG. 3 shows an exemplary computing device in accordance with various embodiments of the disclosure.

FIGS. 4A-4F show representative gating of cells with acquisition set to 1000 basophil events in accordance with various embodiments of the disclosure. FIG. 4A depicts basophil gating; FIG. 4B depicts background control gating for activated basophils (shown as darker circles); FIG. 4C depicts anti-FcεRI control gating for activated basophils (shown as darker circles); FIG. 4D depicts Low Serum Pool gating for activated basophils (shown as darker circles); FIG. 4E depicts intermediate serum pool gating for activated basophils (shown as darker circles); and FIG. 4F depicts high serum pool gating for activated basophils (shown as darker circles).

FIG. 5A shows distribution of % CD63+ basophils during the donor screening phase in an embodiment of the disclosure showing % CD63+ basophils in the screening BAT for a total of 463 donors processed on 15 days over 4 months. Donors selected for the CSU-BAT pool are shown indicated by the word “Yes” over the cell population (darker purple circles), while excluded donors are indicated by the word “No” over the cell population (lighter blue circles).

FIG. 5B shows an additional depiction of basophil donor screening results of FIG. 5A in accordance with an embodiment of the disclosure. The line plot shows % CD63+ basophils in the screening PD-BAT for a total of 463 donors processed on 15 days. Donors selected for the CSU-BAT pool are shown as the dark (purple) shading and excluded donors are shown as lighter blue hatched lines.

FIG. 6 shows reproducibility and precision of PD-BAT in accordance with an embodiment of the disclosure. The inter-assay activated basophil mean (n=4) for the kit controls and the 3 levels (low (LSP), intermediate (ISP), and high (HSP)) of serum pools over 10 different basophil donor pools shows the consistency of PD-BAT. Error bars are one standard deviation from the mean. Shown are background (orange); low serum pool (LSP) (gray); intermediate serum pool (ISP) (blue); high serum pool (HSP) (purple); and the anti-FcεRI positive control (red).

FIG. 7 shows serum temperature stability of the high serum pool. Ambient (blue) and refrigerated (green) temperature conditions for the HSP were tested at 0, 1, 3, 7, and 14 days.

FIG. 8 shows evaluation of the pooled whole blood stability in accordance with an embodiment of the disclosure. Basophil donors were screened and selected. The basophil activation was analyzed for the background, anti-FcεRI, LSP, ISP, and HSP at 0, 2, 6, and 24 hours. The standard deviations ranged from 0.22 (HSP) to 2.23 (anti-FcεRI and HSP).

FIGS. 9A and 9B show a distribution of sera used to determine the technical cut-off. FIG. 9A depicts the distribution of results for 142 specimens with Total IgE<80 IU/L in accordance with an embodiment of the disclosure. Two outliers were eliminated by Chauvenet exclusion method. The central 95% was set as the technical cutoff. FIG. 9B depicts verification of the threshold in a separate analysis of 145 samples negative BHRA results <2.2 CU Indexx (post Chauvenet outlier exclusion) where the upper limit of the reference interval was determined to be 11.52% (10.19 to 13.02, 95% CI).

FIG. 10 shows a method comparison between CU IndexR and PD-BAT in accordance with an embodiment of the disclosure. Quantitative method comparison was completed via EP evaluator (n=343). The regression analysis-Passing-Bablok slope is 0.814. The correlation coefficient is 0.6144. Shown is the PD-BAT technical cut-off (10.61) (horizontal red line); the CU IndexR technical cut-off (>10 CU Index Units) (BHRA technical cut-off) (vertical yellow line); and the Passing-Bablok regression analysis (dashed blue line).

FIGS. 11A, 11B and 11C show 69 serum samples of CSU patients analyzed with the pooled donor method and with three individual donors; with donor 1 shown in FIG. 11A, donor 2 in FIG. 11B and donor 3 shown in FIG. 11C in accordance with an embodiment of the disclosure. Black dashed lines indicate linear regression (R=0.85; R=0.93; R=0.59 respectively for donors 1, 2, and 3.) respectively. Vertical and horizontal lines indicate cut-off of 10.61% CD63+.

FIG. 12 shows PD-BAT comparability of results from two runs in accordance with an embodiment of the disclosure. The two results were plotted against the average of the pair. Donor pool 1 (R=0.997) (violet triangles); green circles, donor pool 2 (R=0.998) (green circles); PD-BAT technical cut-off (horizontal and vertical red lines).

### DETAILED DESCRIPTION

The disclosed subject matter now will be described more fully hereinafter with reference to the accompanying description and drawings, in which some, but not all embodiments of the disclosed subject matter are shown. The disclosed subject matter can be embodied in many different forms and should not be construed as limited to the embodiments set forth herein. Like numbers refer to like elements throughout. Although specific terms are employed herein, they are used in a generic and descriptive sense only and not for purposes of limitation. Additionally, any reference referred to as being “incorporated by reference herein” is to be understood as being incorporated in its entirety.

### Definitions

While the following terms are believed to be well understood by one of ordinary skill in the art, the following definitions are set forth to facilitate explanation of the presently disclosed subject matter. Other definitions are found throughout the specification. Unless otherwise defined, all technical and scientific terms used herein have the same meaning as commonly understood by one of ordinary skill in the art to which this presently described subject matter belongs.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g., 1 to 6.1, and ending with a maximum value of 10 or less, e.g., 5.5 to 10.

As used herein, the singular forms “a,” “an” and “the” are intended to include the plural forms as well, unless the context clearly indicates otherwise. It will be further understood that the terms “comprises” and/or “comprising,” when used in this specification, include the embodiments of “consisting of” and/or “consisting essentially of,” and specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. As used herein, phrases such as “between X and Y” and “between about X and Y” should be interpreted to include X and Y. As used herein, phrases such as “from about X to Y” mean “from about X to about Y.”

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

It will be understood that, although the terms first, second, etc. may be used herein to describe various elements or components, and these elements or components should not be limited by these terms. Also, the sequence of operations (or steps) is not limited to the order presented in the claims or figures unless specifically indicated otherwise.

The term “programmatically” means carried out using a computer program and/or software, processor or ASIC directed operations. The term “electronic” and derivatives thereof refer to automated or semi-automated operations carried out using devices with electrical circuits and/or modules rather than via mental steps and typically refers to operations that are carried out programmatically.

The terms “automated” and “automatic” means that the operations can be carried out with minimal or no manual labor or input. The term “semi-automated” refers to allowing operators some input or activation, but calculations, acquisition, purification, and other steps are done electronically, typically programmatically, without requiring manual input.

The term “about” refers to +/−10% (mean or average) of a specified value or number.

As used herein, a “pool” comprises a plurality (i.e., >1) of samples.

As used herein, a “predetermined threshold” may comprise a range, or a lower limit (e.g., a quantity which determines the lower boundary of a range), or an upper limit (e.g., a quantity which determines the upper boundary of a range). For example, a predetermined threshold may be a range between 2 units, such as 2.00 and 4.00 Such a range would encompass 2.00 and 4.00 and all values in between. A predetermined threshold may also comprise values less than (or alternatively, equal to and/or less than) a unit value (an upper limit). For example, a predetermined threshold may be a range less than or equal to 2.00; this predetermined threshold would encompass all values between and/or equal to 0 and 2.00. A predetermined threshold may also comprise values greater than (or alternatively, equal to and/or greater than) a unit value (a lower limit). For example, a predetermined threshold may be a range greater than or equal to 4.00; this predetermined threshold would encompass all values equal to or greater than the lower limit of 4.00. Unless stated otherwise, a predetermined threshold comprises positive (>0) values.

As used herein, repeatability (or intra-assay precision) describes the closeness of agreement between results of successive measurements of the same analyte and carried out under the same conditions of measurement. Intra-assay repeatability is the measurement of the variability when the same specimen is analyzed during one analytical run.

As used herein reproducibility (or inter-assay precision) describes the closeness of agreement between results of successive measurements of the same analyte and carried out under the same conditions of measurement. Inter-assay repeatability is a measurement of the variability when the same specimen is analyzed during more than one run.

“Sample” or “patient sample” or “biological sample” or “specimen” may be used interchangeably herein. As used herein, biological samples for use in the disclosed pools are distinct from biological samples, e.g., a patient sample or sample from a subject that may be evaluated for the predetermined indication of interest. In some cases, biological samples used for the pool (or pools) are denoted as “donor samples” or “donor biological samples.”

Non-limiting examples of samples that may be used for generating pools and or testing with the disclosed methods and systems include, blood or a blood product (e.g., serum, plasma, or the like). In some cases, such blood products are used directly. The term “blood” encompasses whole blood, blood product or any fraction of blood, such as serum, plasma, buffy coat, or the like as conventionally defined. Other samples may include urine, nasal swabs, a liquid biopsy sample, skin swabs, lesion swabs, or combinations thereof.

The term “individual,” “patient,” or “subject” is used broadly and refers to an individual that provides a sample for testing or analysis and/or an individual who provides a biological sample (e.g., donor biological sample) that may be used for a pool. The “individual” or “patient” or “subject” from whom a sample is collected, obtained, and/or provided by, includes any and all warm-blooded mammalian subjects such as humans and/or animals.

The term “a predetermined indication” is used to describe a medical condition such as a disease and/or a predisposition to a disease. Such diseases may be due to auto-antibodies or other innate medical conditions.

The term “a response indicative of a predetermined indication” is a biological response that can be measured and use to diagnose the presence of or predisposition to the predetermined indication. In certain cases, the response may be cellular in nature as for example, but not limited to basophil activation.

### Abbreviations

ASST, autologous serum skin test
BAT, basophil activation test
BHRA, basophil histamine release assay
CBC, complete blood count
CCR3, CC motif chemokine receptor 3
CD63, cluster of differentiation 63
CSU, chronic spontaneous urticaria
CSU-BAT, indirect basophil activation test by flow cytometry employing a single donor
CV %, coefficient of variation
EDTA, ethylenediaminetetraacetic acid
FcεRI, high-affinity IgE receptor
FSC, forward scatter
HSP, high serum pool
IgE, immunoglobulin E
ISP, intermediate low serum pool
LSP, low serum pool
mAB; monoclonal antibody
NSP, negative serum pool
PD-BAT, pooled donor basophil activation test by flow cytometry
RBC, red blood cell
SD, standard deviation
SSC, side scatter

### Methods

Disclosed are methods to make and use pooled biological samples for use in laboratory and/or clinical testing. In certain embodiments, the method may comprise: screening a plurality of biological samples for a response indicative of the presence of a predetermined indication; and selecting a portion of the plurality of the biological samples that provide the response above a first predetermined threshold to generate the pool.

The pooled biological samples may be further screened to remove samples that have a non-specific response. Thus, in certain embodiments, the method may comprise screening the plurality of biological samples to determine whether the samples have a non-specific response above a predetermined threshold; and removing samples which have a non-specific response from the pool.

For example, the pooled samples may be screened to include samples that have a response above a certain threshold for a positive control. For example, where the pool is going to be used to measure basophil activation using the activation trigger anti-FcεRI mAb and detection of CD63 as a marker of basophil degranulation, the positive control is an anti-FcεRI mAb (e.g., with an appropriate stimulation buffer/mix) and the donors selected for the pool have a response at or above the first predetermined threshold. In an embodiment, donor samples showing a low response (<40% activation) to the anti-FcεRI mAb control are excluded from the pool.

The samples being evaluated for inclusion in the pool may also be screened to exclude donors that exhibit a non-specific response. Thus, the samples screened for use in the pool may be evaluated for a response that occurs upon exposure to a negative control or a reagent that is expected to provide a low response. The negative control may comprise the addition of buffers and other assay reagents such as a low serum pool (LSP) control as disclosed herein. In an embodiment, the predetermined threshold used to determine a non-specific response is different from the first predetermined threshold. In certain embodiments, the two thresholds are the same. In an embodiment, donor samples showing a non-specific activation (>4.6%) by a LSP control are excluded from the pool.

The method may additionally comprise removing samples that do not include cell populations that are typically found in the biological samples used for pooling. For example, where the pool is used to screen for basophil activation as discussed herein, biological samples that do not include cell types such as lymphocytes, monocytes or granulocytes may be excluded. In certain embodiments, where the pool is used to measure basophil activation as disclosed herein, biological samples may be excluded from the pool if the basophil population is not clearly distinguishable between SSC and CCR3 to prevent inadequate gating and results.

Depending on the response that is of interest, the biological samples may vary. In certain embodiments, the biological samples used for pooling are blood. For example, in certain embodiments, blood samples collected for complete blood count (CBC) screening may be used. Such samples may be used after storage. In certain embodiments, individual donor blood is stored at room temperature up to 24 hours post collection. Only blood within 24 hours post collection is screened in the assay. After blood donors are screened, individual aliquots of blood are stored in the refrigerator up to an additional 24 hours when refrigerated (4° C.) before the blood is pooled. In an embodiment, blood samples are processed to provide white blood cells. In an embodiment, such white blood cells include basophils. Or, other samples (e.g., plasma, serum, saliva) may be used for pooling.

Pooled samples may be useful for cellular-based testing such as, but not limited to, flow cytometry. For example, in some cases pooled blood samples are used to test a patient serum sample for a disease caused by abnormal basophil activation. Thus, in certain embodiments, the samples being evaluated for inclusion in the pool are screened for basophil activation. In an embodiment, the samples being evaluated for inclusion in the pool are screened for basophil activation using anti-FcεRI mAb as an activation trigger and detection of CD63 as a marker of basophil degranulation. In an embodiment, the predetermined indication is an auto-antibody disease. In an embodiment, the method may then further comprise using the pooled samples to test patient sera for a basophil response indicative of chronic spontaneous urticaria.

In an embodiment, the pooled samples are used to evaluate a response generated by exposure to a separate sample from a subject. For example, in an embodiment, the pooled samples are used to evaluate basophil activation that occurs in the pooled samples upon exposure of the pooled samples to a biological sample from a test subject. In an embodiment, the biological sample from the test subject is serum.

The pool size may depend on the testing being performed as well as the availability of samples for pooling. In certain embodiments, the pool comprises at least 10, or at least 20, or at least 30 biological samples. Or more or fewer samples may be used. The pooled samples are derived from a larger collection of samples. For example, in certain embodiments, the pool may comprise about 50% or more of the samples that are screened for pooling. Or larger or smaller percentages of the samples available for screening may be used.

Pooling of the samples allows for development of a robust test for various indications as the pooled samples may provide a more consistent response than individual samples. In certain embodiments, the technical cutoff for the pooled samples as indicating a positive response indicative of the presence of a predetermined indication is defined using a 95% confidence interval (CI) of a plurality of normal samples. Thus, as shown in Examples 2 and 3, pooling samples for use in testing can provide a significant improvement over using individual samples.

In an embodiment, the precision of the pool is evaluated by comparing results to known quality control (QC) samples. In an embodiment, the known quality controls comprise: (i) a biological sample that generates a high response at or above a first predetermined QC threshold (i.e., a high QC); (ii) a biological sample that generates an intermediate response above a second predetermined QC threshold but lower than the first QC threshold (i.e., an intermediate QC); and (iii) a biological sample that generates a low response at or below a third predetermined QC threshold which is lower than the second QC threshold (i.e., a low QC). The thresholds used for QC may be different than the thresholds for evaluation of high responders to the positive anti-FcεRI mAb control and the threshold used to determine a non-specific response. Or some of the thresholds may be the same.

In an embodiment, the high, intermediate and low quality controls comprise serum. Also, in certain embodiments, the high, intermediate and low quality controls comprise pools of samples. For example, the high QC may comprise pooled serum that generates a high response at or above the first predetermined QC threshold (i.e., a High Serum Pool; HSP), the intermediate (QC) may comprise pooled serum that generates an intermediate response above a second predetermined QC threshold but lower than the first threshold (i.e., an Intermediate Serum Pool; ISP), and the low QC may comprise pooled serum that generates a low response at or below a third predetermined QC threshold which is lower than the second threshold (i.e., a Low Serum Pool; LSP). The thresholds used for QC may be different than the thresholds for evaluation of high responders to the positive anti-FcεRI mAb control and the threshold used to determine a non-specific response. Or some of the thresholds may be the same.

The quality controls may also comprise a known positive control and/or a known negative control. For example, where the pool is used to measure basophil activation using the activation trigger anti-FcεRI mAb and detection of CD63 as a marker of basophil degranulation, the positive control is an anti-FcεRI mAb and a stimulation buffer as disclosed herein. The negative control may comprise the addition of buffers and/or other assay reagents.

The predetermined QC thresholds used for the QC pools may be based on a predetermined reference value that relates to the response being measured. For example, in certain embodiments the high QC (e.g., HSP) may generate a response that is ≥3.0 standard deviations (SD) above a defined technical cut-off for the response being measured. The intermediate QC (e.g., ISP) may generate a response that is 1 to 3 SD below the technical cut-off for the response being measured. The low QC (e.g., LSP) may generate a response that is >3 SD below the technical cut-off for the response being measured.

For example, for assessment of basophil activation, a Low Serum Pool (LSP) Quality Control may comprise a pool of previously screened and stored sera with results between 0-4.67% activated basophils, i.e., about 3 standard deviations (SD) below the reference interval (the PD-BAT technical cut-off) of 10.61. Also, in certain embodiments, the Intermediate Serum Pool (ISP) Quality Control may comprise a pool of previously screened and stored sera with results between 4.67-8.63% activated basophils, i.e. between about 1 to 3 SD below the reference interval (the PD-BAT technical cut-off) of 10.61. Also, in certain embodiments, the High Serum Pool Control (HSP) may comprise a pool of previously screened and stored serum with results greater than 16.55% activated basophils, i.e., about 3 SD above the reference interval (the PD-BAT technical cut-off) of 10.61. Sub-aliquots of these serum control pools may be stored at −20° C. for future use.

Also disclosed are methods of using pooled samples for testing whether a subject, e.g., a patient, has a predetermined indication of interest. Thus, disclosed is a method to test for a response indicative of the presence of a predetermined indication comprising in a sample from a subject comprising the steps of: generating a plurality of pooled samples that provide the response above a first predetermined threshold and optionally, do not exhibit a non-specific response; and adding a portion of the sample from the subject to the pooled samples to determine if the sample generates the response.

The pooled samples used for testing may be screened to remove samples that are not responsive to a positive control and/or have a non-specific response. Thus, in certain embodiments, the method may comprise screening the plurality of samples to determine whether the samples have a non-specific response above a predetermined threshold; and removing samples which have a non-specific response from the pool.

For example, the pooled samples may be screened to remove samples that have a response below a certain threshold for a positive control. For example, where the pool is going to be used to measure basophil activation using the activation trigger anti-FcεRI mAb and detection of CD63 as a marker of basophil degranulation, the positive control is an anti-FcεRI mAb and a stimulation buffer as disclosed herein and the donors selected for the pool have a response at or above the first predetermined threshold. In an embodiment, donor samples showing a low response (<40% activation) to the anti-FcεRI mAb control are excluded from the pool.

The negative control may comprise the addition of buffers and other assay reagents such as a low serum pool (LSP) control as disclosed herein. In an embodiment, the predetermined threshold used to determine a non-specific response is different from the first predetermined threshold. In certain embodiments, the two thresholds are the same. In an embodiment, donor samples showing a non-specific activation (>4.6%) by a LSP control are excluded from the pool.

The method may additionally comprise removing samples that do not include cell populations that are typically found in the biological samples used for pooling. For example, biological samples that do not include cell types such as lymphocytes, monocytes or granulocytes may be excluded. In certain embodiments, where the pool is used to measure basophil activation as disclosed herein, biological samples may be excluded from the pool if the basophil population is not clearly distinguishable between SSC and CCR3 to prevent inadequate gating and results.

Depending on the response that is of interest, the biological samples used to generate the pool may vary. In certain embodiments, the biological samples used for pooling are blood. For example, in certain embodiments, blood samples collected for complete blood count (CBC) screening may be used. Such samples may be used after storage. For example, individual donor blood is stored at room temperature up to 24 hours post collection. Only blood within 24 hours post collection is screened in the assay. After blood donors are screened, individual aliquots of blood are stored in the refrigerator up to an additional 24 hours when refrigerated (4° C.) before the blood is pooled. In an embodiment, the blood samples are processed to provide white blood cells. In an embodiment, such white blood cells include basophils. Or, other samples (e.g., plasma, serum, saliva) may be used for pooling.

The sample from the test subject (e.g., patient) is distinct from the samples used for the pool. In an embodiment, the biological sample from the test subject is serum. Or other types of patient samples may be tested with the pool.

Pooled samples may be useful for cellular-based testing such as, but not limited to, flow cytometry. For example, in some cases pooled blood samples are used to test a patient serum sample for a disease caused by abnormal basophil activation. Thus, in certain embodiments, the samples being evaluated for inclusion in the pool are screened for basophil activation. In an embodiment, the samples being evaluated for inclusion in the pool are screened for basophil activation using the activation trigger anti-FcεRI mAb and detection of CD63 as a marker of basophil degranulation. In an embodiment, the predetermined indication is an auto-antibody disease. In an embodiment, the method may comprise using the pooled samples to test patient sera for a basophil response indicative of chronic spontaneous urticaria.

The pool size may depend on the testing being performed as well as the availability of samples for pooling. In certain embodiments, the pool used for testing comprises at least 10, or at least 20, or at least 30 biological samples. Or more or fewer samples may be used. The pooled samples are derived from a larger collection of samples. For example, in certain embodiments, the pool may comprise about 50% or more of the samples that are screened for pooling. Or larger or smaller percentages of the samples available for screening may be used.

As shown in Examples 2 and 3 herein, pooling of the samples allows for development of a robust test for various indications as the pooled samples may provide a more consistent response than individual samples. In certain embodiments, the technical cutoff for the pooled samples as indicating a positive response indicative of the presence of a predetermined indication is defined using a 95% CI of a plurality of normal samples.

As discussed above, the precision of the pool used for testing may be evaluated by comparing results using the patient sample to known quality control (QC) samples. In an embodiment, the known quality controls comprise: (i) a biological sample that generates a high response at or above the first predetermined QC threshold (high QC); (ii) a biological sample that generates an intermediate response above a second predetermined QC threshold but lower than the first threshold (intermediate QC); and (iii) a biological sample that generates a low response at or below a third predetermined QC threshold which is lower than the second threshold (low QC). The thresholds used for QC may be different than the thresholds for evaluation of high responders to the positive anti-FcεRI mAb control and the threshold used to determine a non-specific response.

In an embodiment, the high, intermediate and low quality controls comprise serum. Also, in certain embodiments, the high, intermediate and low quality controls comprise pools of samples. For example, the high QC may comprise pooled serum that generates a high response at or above the first predetermined QC threshold (i.e., a High Serum Pool; HSP), the intermediate (QC) may comprise pooled serum that generates an intermediate response above a second predetermined QC threshold but lower than the first threshold (i.e., an Intermediate Serum Pool; ISP), and the low QC may comprise pooled serum that generates a low response at or below a third predetermined QC threshold which is lower than the second threshold (i.e., a Low Serum Pool; LSP). The quality controls may also comprise a known positive control and/or a known negative control. For example, where the pool is used to measure basophil activation using the activation trigger anti-FcεRI mAb and detection of CD63 as a marker of basophil degranulation, the positive control is an anti-FcεRI mAb. The negative control may comprise the addition of buffers and other assay reagents.

The predetermined QC thresholds used for the QC pools may be based on a predetermined reference value that relates to the response being measured. In certain embodiments the high QC may have generate a response that is ≥3.0 standard deviations (SD) above a defined technical cut-off for the response being measured. The intermediate QC may generate a response that is 1 to 3 SD below the technical cut-off for the response being measured. The low QC may generate a response that is >3 SD below the technical cut-off for the response being measured.

For example, for assessment of basophil activation in a sample from a subject (e.g., patient), a Low Serum Pool (LSP) Quality Control (LSP) may comprise a pool of previously screened and stored sera with results between 0-4.67% activated basophils, i.e., about 3 standard deviations (SD) below the reference interval (the PD-BAT technical cut-off) of 10.61. Also, in certain embodiments, the Intermediate Serum Pool (ISP) Quality Control may comprise a pool of previously screened and stored sera with results between 4.67-8.63% activated basophils, i.e. between about 1 to 3 SD below the reference interval (the PD-BAT technical cut-off) of 10.61. In certain embodiments, the High Serum Pool Control (HSP) may comprise a pool of previously screened and stored serum with results greater than 16.55% activated basophils, i.e., about 3 SD above the reference interval (the PD-BAT technical cut-off) of 10.61. Sub-aliquots of these serum control pools may be stored at −20° C. for future use.

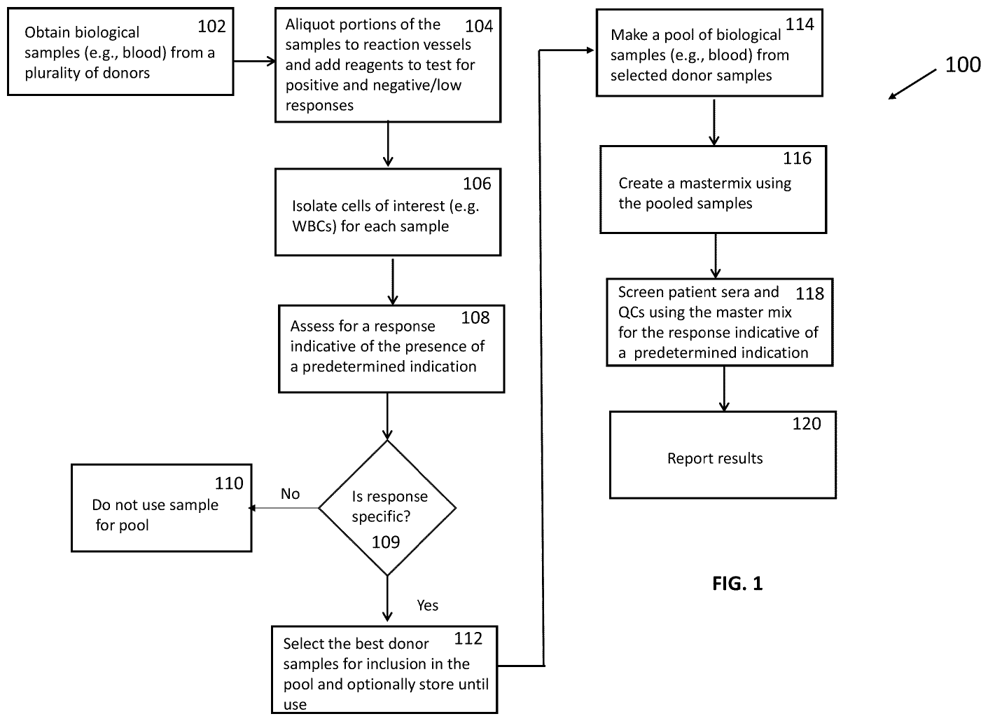

FIG. 1 shows an illustration of a method 100 of the disclosure. As shown, the method may comprise the step 102 of obtaining biological samples from each of a plurality of donors. In an embodiment, blood or a blood product (e.g., serum) is used. The method may further comprise the step 104 of aliquoting portions of each donor sample into reaction vessels and adding reagents to assess the individual donor samples for a response indicative of a predetermined indication. For example, to each tube may be added reagents to assess whether the donor sample has a sufficient high response to a positive control and/or does not have a non-specific response when assessed using a negative or low response control. Where the response being measured is basophil activation, the reagents may include anti-FcεRI as a positive control and LSP master mixes and/or buffers to separate donor samples as two separate negative controls (i.e., to assess for donor samples that generate non-specific basophil activation).

Next the method may include the steps of isolating cells from the donor samples 106 and assessing the cells for a response indicative of a predetermined indication 108. As disclosed herein, in certain embodiments the method may comprise the individual steps of a heated incubation at 37° C. for stimulating basophil activation in response to a stimulant (i.e., anti-FcεRI, or LSP), lysing red blood cells (RBCs), centrifugation and removal of the supernatant, and resuspension of pelleted white blood cells (WBCs) including basophils. The samples may then be acquired in a flow cytometer and gated as disclosed herein. As disclosed herein, where the predetermined indication is chronic spontaneous urticaria the response being measured is basophil activation measured using the activation trigger anti-FcεRI mAb detected as CD63+ expression.

In an embodiment, donor samples are each assessed 109 to determine if the response is specific. If the response is not specific, the donor sample is not used for the pool 110. If the response is specific, the donor sample is selected for use in the pool 112.

For example, for a basophil activation test, donors that have high anti-FcεRI mAb stimulation and that are not highly responsive to a low serum pool (LSP) or do not show high background may be included in the pool. Conversely, in an embodiment, donors showing a non-specific activation (>4.6%) by a low serum pool (LSP; i.e., low QC pool) are excluded from being used for the pool. Also, donors showing a low response (<40%) to an anti-FcεRI mAb positive control are excluded from being used in the pool. Donors may also be excluded if in the SSC singlets plot one of the following populations is missing: lymphocytes, monocytes, or granulocytes. Donors may also be excluded if they do not have at least 300 basophil events within 200 seconds of acquisition and/or if the basophil population is not clearly distinguishable between SSC and CCR3 to prevent inadequate gating and results. Once selected for use in the pool, a plurality of the donor samples (e.g., about 16-20) are selected for inclusion in the pool. The selected donor samples may be stored (e.g., refrigerated overnight) prior to pooling and use in an assay of a patient sample.

Next, aliquots of each of the selected donor samples are pooled 114 for use in a assay to test a patient sample to determine if the patient sample generates a response indicative of the predetermined indication. At this point, a mastermix is created using the pooled donor samples 116. This mastermix may then be used to assess a patient sample, as well as suitable quality controls (e.g., LSP, ISP, HSP, background and positive controls) for the response being measured 118. This screening may include the steps of a heated incubation to stimulate basophil response, lysing the red blood cells (RBCs) in the pooled donor sample, centrifugation and removal of the supernatant, and resuspension of pelleted white blood cells (WBCs) including basophils. The samples may then be acquired in a flow cytometer and gated. The results may then be reported 120 to the subject and/or his or her health care provider.

### Compositions and Kits

Also disclosed are compositions and kits for performing the disclosed methods. Thus, in an embodiment, disclosed is a composition comprising a pool of biological samples for screening a sample from a subject for a predetermined indication, wherein the pool comprises a plurality biological samples each exhibiting a response above a first predetermined threshold and optionally do not exhibit a non-specific response, and wherein the response is indicative of the presence of the predetermined indication. In an embodiment, the composition comprises individually packaged pooled donor biological samples.

Also disclosed are kits comprising a composition comprising a pool of biological samples for screening a sample from a subject for a predetermined indication, wherein the pool comprises a plurality biological samples each exhibiting a response above a first predetermined threshold and optionally do not exhibit a non-specific response, wherein the response is indicative of the presence of the predetermined indication and instructions for use. In an embodiment, the composition used in the kit comprises individually packaged/packed pooled donor biological samples.

The pooled samples used for either the composition and/or the kit may be screened to remove samples that have a non-specific response. Thus, in certain embodiments, the plurality of samples used for the pool are screened to determine whether the samples have a non-specific response above a predetermined threshold and samples that exhibit a non-specific response are not used from the pool.

For example, the pooled samples may be screened to remove samples that have a response below a certain threshold for a positive control. In an embodiment, where the pool is going to be used to measure basophil activation using the activation trigger anti-FcεRI mAb and detection of CD63 as a marker of basophil degranulation, the positive control is an anti-FcεRI mAb and a stimulation buffer and the donors selected for the pool have a response at or above the first predetermined threshold. In an embodiment, donor samples showing a low response (<40% activation) to the anti-FcεRI mAb control are excluded from the pool.

The negative control may comprise the addition of buffers and other assay reagents such as a low serum pool (LSP) control as disclosed herein. In an embodiment, the predetermined threshold used to determine a non-specific response is different from the first predetermined threshold. In certain embodiments, the two thresholds are the same. In certain embodiments, the two thresholds are the same. In an embodiment, donor samples showing a non-specific activation (>4.6%) by a LSP control are excluded from the pool.

In certain embodiments, samples that do not include cell populations that are typically found in the biological samples are excluded from the pool. For example, where the pool is used to screen for basophil activation as discussed herein, biological samples that do not include cell types such as lymphocytes, monocytes or granulocytes may be excluded. In certain embodiments, where the pool is used to measure basophil activation as disclosed herein, biological samples may be excluded from the pool if the basophil population is not clearly distinguishable between SSC and CCR3 to prevent inadequate gating and results.

Depending on the response that is of interest, the biological samples used for the pools of the compositions and kits may vary. In certain embodiments, the biological samples used for pooling are blood. For example, in certain embodiments, blood samples collected for complete blood count (CBC) screening may be used. Such samples may be used after storage. For example, individual donor blood is stored at room temperature up to 24 hours post collection. Only blood within 24 hours post collection is screened in the assay. After blood donors are screened, individual aliquots of blood are stored in the refrigerator up to an additional 24 hours when refrigerated (4° C.) before the blood is pooled. In an embodiment, blood samples are processed to provide white blood cells. In an embodiment, such white blood cells include basophils. Or, other samples (e.g., plasma, serum, saliva) may be used for pooling.

Pooled samples may be useful for cellular-based testing such as, but not limited to, flow cytometry. For example, in some cases pooled blood samples of the disclosed compositions and kits may be used to test a patient serum sample for a disease caused by abnormal basophil activation. Thus, in certain embodiments, the samples being evaluated for inclusion in the pool are screened for basophil activation. In an embodiment, the samples being evaluated for inclusion in the pool are screened for basophil activation using anti-FcεRI mAb as an activation trigger and detection of CD63 as a marker of basophil degranulation. In an embodiment, the predetermined indication is an auto-antibody disease. In an embodiment, the pooled samples of the compositions and kits may be used to test patient sera for a basophil response indicative of chronic spontaneous urticaria.

In an embodiment, the pooled samples of the compositions and/or kits are used to evaluate a response generated by exposure to a separate sample from a subject. For example, in an embodiment, the pooled samples are used to evaluate basophil activation that occurs in the pooled samples upon exposure to a biological sample from a test subject. In an embodiment, the biological sample from the test subject is serum.

The pool size of the composition and/or kit may depend on the testing being performed as well as the availability of samples for pooling. In certain embodiments, the pool comprises at least 10, or at least 20, or at least 30 biological samples. Or more or fewer samples may be used. The pooled samples may be derived from a larger collection of samples. For example, in certain embodiments, the pool may comprise about 50% or more of the samples that are screened for pooling. Or larger or smaller percentages of the samples available for screening may be used.

Pooling of the samples allows for development of a robust test for various indications as the pooled samples may provide a more consistent response than individual samples. In certain embodiments, the technical cutoff for the pooled samples used in the compositions and/or kits as indicating a positive response indicative of the presence of a predetermined indication is defined using a 95% confidence interval (CI) of a plurality of normal samples.

In an embodiment, the precision of the pool of the compositions and/or kits is evaluated by comparing results to known quality control (QC) samples. In an embodiment, the known quality controls comprise: (i) a biological sample that generates a high response at or above the first predetermined QC threshold (high QC); (ii) a biological sample that generates an intermediate response above a second predetermined QC threshold but lower than the first threshold (intermediate QC); and (iii) a biological sample that generates a low response at or below a third predetermined QC threshold which is lower than the second threshold (low QC). The thresholds used for QC may be different than the thresholds for evaluation of high responders to the positive anti-FcεRI mAb control and the threshold used to determine a non-specific response.

In an embodiment, the high, intermediate and low quality controls comprise serum. Also, in certain embodiments, the high, intermediate and low quality controls comprise pools of samples. For example, the high QC may comprise pooled serum that generates a high response at or above the first predetermined QC threshold (HSP), the intermediate (QC) may comprise pooled serum that generates an intermediate response above a second predetermined QC threshold but lower than the first threshold (ISP), and the low QC may comprise pooled serum that generates a low response at or below a third predetermined QC threshold which is lower than the second threshold (LSP). The quality controls may also comprise a known positive control and/or a known negative control. For example, where the pool is used to measure basophil activation using the activation trigger anti-FcεRI mAb and detection of CD63 as a marker of basophil degranulation, the positive control is an anti-FcεRI mAb and a stimulation buffer. The negative control may comprise the addition of buffers and other assay reagents.

The predetermined thresholds used for the QC pools may be based on a predetermined reference value that relates to the response being measured. For example, in certain embodiments the high QC may have generate a response that is ≥3.0 standard deviations (SD) above a defined technical cut-off for the response being measured. The intermediate QC may generate a response that is 1 to 3 SD below the technical cut-off for the response being measured. The low QC may generate a response that is >3 SD below the technical cut-off for the response being measured.

For example, for compositions and/or kits used for assessment of basophil activation, a Low Serum Pool (LSP) Quality Control (LSP) may comprise a pool of previously screened and stored sera with results between 0-4.67% activated basophils, i.e., about 3 standard deviations (SD) below the reference interval (the PD-BAT technical cut-off) of 10.61. Also, in certain embodiments, the Intermediate Serum Pool (ISP) Quality Control may comprise a pool of previously screened and stored sera with results between 4.67-8.63% activated basophils, i.e. between about 1 to 3 SD below the reference interval (the PD-BAT technical cut-off) of 10.61. In certain embodiments, the High Serum Pool Control (HSP) may comprise a pool of previously screened and stored serum with results greater than 16.55% activated basophils, i.e., about 3 SD above the reference interval (the PD-BAT technical cut-off) of 10.61. Sub-aliquots of these serum control pools may be stored at −20° C. for future use.

### Systems

Also disclosed are systems for performing the disclosed methods and/or using any of the disclosed compositions and kits. The disclosed systems may comprise certain stations (physical locations) and/or components (e.g., laboratory reagents). In some embodiments, the stations and or components are each separate from each other. In some embodiments, the stations and/or components are combined or are the same.

Thus, disclosed is a system for generating a pool of biological samples for use in testing comprising: a component or station for screening a plurality of biological samples for a response indicative of the presence of a predetermined indication; and a component or station for selecting a plurality of the samples that provide the response above a first predetermined threshold to generate the pool. The system may further comprise a component or station for assessing a biological sample from a subject for the response, the biological sample from the subject being distinct from any of the biological samples used to generate the pool, to determine if the sample generates the response and to quantify the level of the response as compared to the pools.

Also, in certain embodiments, the system may further comprise a station and/or component for: screening the plurality of samples to determine whether the samples have a non-specific response; and removing samples which have a non-specific response from the pool as disclosed herein. In an embodiment, the predetermined threshold used to determine a non-specific response is different from the predetermined threshold used to generate the pool. In certain embodiments, the two thresholds are the same.

For example, the system may comprise a station and/or component to screen pooled samples to select for samples that have a response above a predetermined threshold for a positive control and/or to remove samples that have a non-specific response. Thus, the system may have a station and/or component to add a positive control as well as a negative control (e.g., buffer or a LSP) to certain of the biological samples that are to be screened for pooling. For example, where the pool is going to be used to measure basophil activation using the activation trigger anti-FcεRI mAb and detection of CD63 as a marker of basophil degranulation, the system may comprise a component to add an anti-FcεRI mAb as a positive control and a component to determine which the donors have a non-specific response (e.g., to a negative control or LSP).

The system may further comprise a component to select such donors for the pool. In an embodiment, the predetermined threshold used to determine a non-specific response is different from the first predetermined threshold. In certain embodiments, the two thresholds are the same. In an embodiment, donor samples showing a low response (<40% activation) to the anti-FcεRI mAb control are excluded from the pool and/or donor samples showing a non-specific activation (>4.6%) by a LSP control are excluded from the pool. The system may further comprise a component and/or station to assess whether the biological samples being evaluated for inclusion in the pool do not include cell populations that are typically found in the biological samples used for pooling. For example, where the pool is used to screen for basophil activation as discussed herein, biological samples that do not include cell types such as lymphocytes, monocytes or granulocytes may be excluded. In certain embodiments, where the pool is used to measure basophil activation as disclosed herein, biological samples may be excluded from the pool if the basophil population is not clearly distinguishable between SSC and CCR3 to prevent inadequate gating and results.

Depending on the response that is of interest, the biological samples used for the pool may vary. In certain embodiments, the biological samples used for pooling are blood. For example, in certain embodiments, blood samples collected for complete blood count (CBC) screening may be used. Such samples may be used after storage. For example, individual donor blood is stored at room temperature up to 24 hours post collection. Only blood within 24 hours post collection is screened in the assay. After blood donors are screened, individual aliquots of blood are stored in the refrigerator up to an additional 24 hours when refrigerated (4° C.) before the blood is pooled. In an embodiment, blood samples are processed to provide white blood cells. In an embodiment, such white blood cells include basophils. Or, other samples (e.g., plasma, serum, saliva) may be used for pooling.

Pooled samples may be useful for cellular-based testing such as, but not limited to, flow cytometry. For example, in some cases pooled blood samples are used to test a patient serum sample for a disease caused by abnormal basophil activation. Thus, in certain embodiments, the samples being evaluated for inclusion in the pool are screened for basophil activation. In an embodiment, the samples being evaluated for inclusion in the pool are screened for basophil activation using anti-FcεRI mAb as an activation trigger and detection of CD63 as a marker of basophil degranulation. In an embodiment, the predetermined indication is an auto-antibody disease. In an embodiment, the method may then further comprise using the pooled samples to test patient sera for a basophil response indicative of chronic spontaneous urticaria.

In an embodiment, the system may comprise a station or component for using the pooled samples to evaluate a response generated by exposure to a separate sample from a subject. For example, in an embodiment, the pooled samples are used to evaluate basophil activation that occurs in the pooled samples upon exposure to a biological sample from a test subject. In an embodiment, the biological sample from the test subject is serum.

The pool size used in the disclosed system may depend on the testing being performed as well as the availability of samples for pooling. In certain embodiments, the pool comprises at least 10, or at least 20, or at least 30 biological samples. Or more or fewer samples may be used. The pooled samples are derived from a larger collection of samples. For example, in certain embodiments, the pool may comprise about 50% or more of the samples that are screened for pooling. Or larger or smaller percentages of the samples available for screening may be used.

Pooling of the samples allows for development of a robust test for various indications as the pooled samples may provide a more consistent response than individual samples. In certain embodiments, the system may comprise a station and/or component to evaluate if the technical cutoff for the pooled samples as indicating a positive response indicative of the presence of a predetermined indication as defined using a 95% confidence interval (CI) of a plurality of normal samples.

In an embodiment, the precision of the pool is evaluated by comparing results to known quality control (QC) samples. Thus, the disclosed systems may comprise such QC samples. In an embodiment, the known quality controls comprise: (i) a biological sample that generates a high response at or above the first predetermined QC threshold (high QC); (ii) a biological sample that generates an intermediate response above a second predetermined QC threshold but lower than the first threshold (intermediate QC); and (iii) a biological sample that generates a low response at or below a third predetermined QC threshold which is lower than the second threshold (low QC). The thresholds used for QC may be different than the thresholds for evaluation of high responders to the positive anti-FcεRI mAb control and the threshold used to determine a non-specific response.

In an embodiment, the high, intermediate and low quality controls comprise serum. Also, in certain embodiments, the high, intermediate and low quality controls comprise pools of samples. For example, the high QC may comprise pooled serum that generates a high response at or above a first predetermined QC threshold (HSP), the intermediate (QC) may comprise pooled serum that generates an intermediate response above a second predetermined QC threshold but lower than the first QC threshold (ISP), and the low QC may comprise pooled serum that generates a low response at or below a third predetermined QC threshold which is lower than the second QC threshold (LSP). The quality controls may also comprise a known positive control and/or a known negative control. For example, where the pool is used to measure basophil activation using the activation trigger anti-FcεRI mAb and detection of CD63 as a marker of basophil degranulation, the positive control is an anti-FcεRI mAb and a stimulation buffer as disclosed herein. The negative control may comprise the addition of buffers and other assay reagents.

The predetermined QC thresholds used for the QC pools may be based on a predetermined reference value that relates to the response being measured. For example, in certain embodiments the high QC may have generate a response that is ≥3.0 standard deviations (SD) above a defined technical cut-off for the response being measured. The intermediate QC may generate a response that is 1 to 3 SD below the technical cut-off for the response being measured. The low QC may generate a response that is >3 SD below the technical cut-off for the response being measured.

For example, for assessment of basophil activation, a Low Serum Pool (LSP) Quality Control (LSP) may comprise a pool of previously screened and stored sera with results between 0-4.67% activated basophils, i.e., about 3 standard deviations (SD) below the reference interval (the PD-BAT technical cut-off) of 10.61. Also, in certain embodiments, the Intermediate Serum Pool (ISP) Quality Control may comprise a pool of previously screened and stored sera with results between 4.67-8.63% activated basophils, i.e. between about 1 to 3 SD below the reference interval (the PD-BAT technical cut-off) of 10.61. In certain embodiments, the High Serum Pool Control (HSP) may comprise a pool of previously screened and stored serum with results greater than 16.55% activated basophils, i.e., about 3 SD above the reference interval (the PD-BAT technical cut-off) of 10.61. Sub-aliquots of these serum control pools may be stored at −20° C. for future use with the system.

In an embodiment, the system for testing samples may be automated. For example, in certain embodiments, the system may comprise a computer-program product tangibly embodied in a non-transitory machine-readable storage medium including instructions configured to run any of the components or stations of the system.

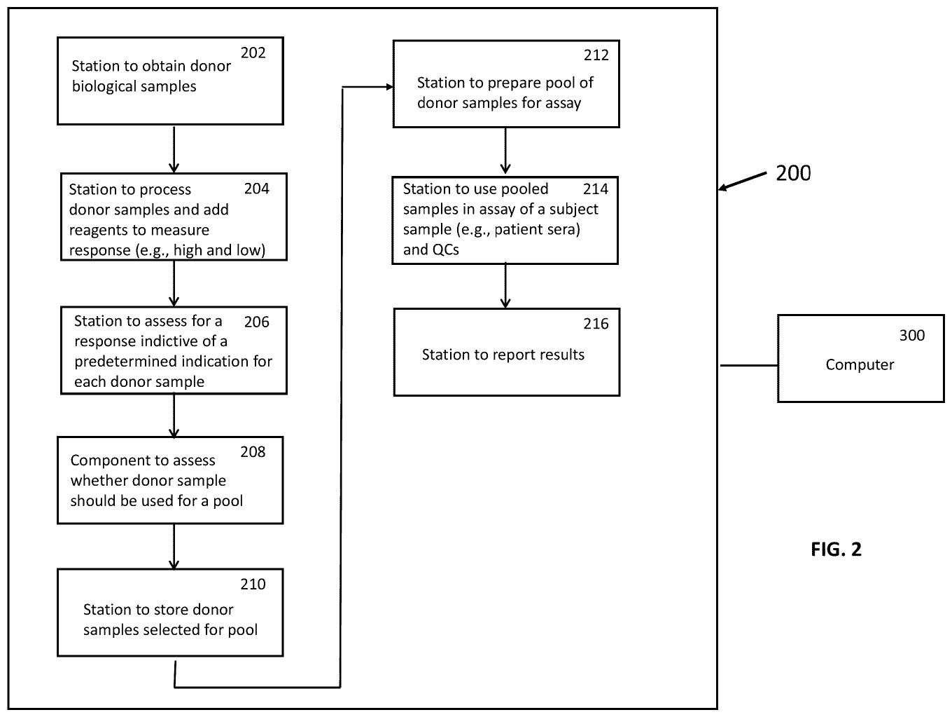

FIG. 2 shows an illustration of a system 200 of the disclosure. As shown, the system may comprise a station and/or component 202 for donor biological samples to be evaluated for inclusion in a pool. In an embodiment, blood or a blood product (e.g., serum) is used. The system may further comprise a station and/or component 204 to process the donor samples. This station and/or component may comprise reagents that are added to measure a response that is indicative of a predetermined indication. For example, portions of each donor sample may be aliquoted into reaction vessels and reagents added to assess the individual donor samples for a response indicative of a predetermined indication. For example, to each tube may be added reagents to assess whether the donor sample has a sufficient high response to a positive control and/or does not have a non-specific response when assessed using a negative or low response control. Where the response being measured is basophil activation, the reagents may include anti-FcεRI as a positive control and LSP master mixes and/or buffers to separate donor samples as two separate negative controls (i.e., to assess for donor samples that generate non-specific basophil activation). The system may also include a station and/or component to isolate cells from each donor samples. As disclosed herein, in certain embodiments that may comprise a component(s) and/or station(s) for lysing red blood cells (RBCs), centrifugation and removal of the supernatant, and resuspension of pelleted white blood cells (WBCs) including basophils.

The system may further comprise a station and/or component 206 and assessing the cells for a response indicative of a predetermined indication. For example, in certain embodiments, the cells are assessed by flow cytometry. The samples may thus be acquired in a flow cytometer and gated as disclosed herein. As disclosed herein, where the predetermined indication is chronic spontaneous urticaria the response being measured is basophil activation measured using the activation trigger anti-FcεRI mAb detected as CD63+ expression.

The system may also comprise a station and/or component to assess whether the donor samples should be used for the pool 208. As disclosed herein, such assessment may include an evaluation of the donor cells' ability to generate the response when exposed to a positive control and/or a negative control such as buffer or a low serum pool (LSP). As disclosed herein, where the predetermined indication is chronic spontaneous urticaria the response being measured is basophil activation measured using the activation trigger anti-FcεRI mAb detected as CD63+ expression. If the response to the positive control is not above a certain threshold, or if the response to the background or LSP is above a certain threshold (i.e., is not specific), the donor sample is not used for the pool or pools. For example, in certain embodiments, for a basophil activation test donors that have high FcεRI stimulation and that are not highly responsive to LSP are selected for inclusion in the pool. Thus, donors may be excluded if they are non-responders, i.e., any donor that has <10-40% activated basophils for the FcεRI control. Donors may also be excluded if in the SSC singlets plot one of the following populations is missing: lymphocytes, monocytes, granulocytes. Donors may also be excluded if they do not have at least 300 basophil events within 200 seconds of acquisition.

The system may include a station and/or component 212 to prepare a pool of selected donor samples to be used to evaluate the response generated by a separate patient sample. Thus, the system may also include a station and/or component to assess a sample from another subject (i.e., a patient seeking a diagnosis) for the predetermined indication using the established pools 212. As disclosed herein, in certain embodiments, the predetermined indication is chronic spontaneous urticaria. Thus, in such embodiments, this station may comprise a component and/or components for assessing a biological sample from a subject for the response, the biological sample from the subject being distinct from any of the biological samples used to generate the pools, to determine if the sample generates the response and to quantify the level of the response.

This station may further comprise components and/or reagents to compare the results obtained with the patent sample to a positive control, a negative control and high, intermediate and low QC controls (e.g., HSP, ISP and LSP) as disclosed herein. The system may also include a station and/or component to report results to the subject and/or his or her health care provider 216.

As illustrated in FIG. 2, any of the stations and/or components of the system may be automated, robotically controlled, and/or controlled at least in part by a computer 300 and/or programmable software. Thus, the system may comprise a computer-program product tangibly embodied in a non-transitory machine-readable storage medium, including instructions configured to run the system or any part (e.g., station or component) of the system and/or perform a step or steps of the methods of any of the disclosed embodiments. In some embodiments, a system is provided that includes one or more data processors and a non-transitory computer readable storage medium containing instructions which, when executed on the one or more data processors, cause the one or more data processors to perform part or all of one or more methods or processes disclosed herein and/or run any of the parts of the systems disclosed herein.

Also disclosed is a computer-program product tangibly embodied in a non-transitory machine-readable storage medium including instructions configured to cause one or more data processors to run any of the components of a system or to perform any of the steps of the disclosed methods. Any suitable computer readable medium may be utilized including hard disks, CD-ROMs, optical storage devices, or magnetic storage devices.

Thus, in an embodiment, disclosed a computer-program product tangibly embodied in a non-transitory machine-readable storage medium including instructions configured to: screen a plurality of biological samples for a response indicative of the presence of a predetermined indication; and select a plurality of the samples that provide the response above a first predetermined threshold to generate the pool.

Also disclosed a computer-program product tangibly embodied in a non-transitory machine-readable storage medium including instructions configured to test for a response indicative of the presence of a predetermined indication comprising in a sample from a subject wherein the method comprises: generating a plurality of pools of biological samples, wherein each pool provides a response above a predetermined threshold that is indicative of the presence of the predetermined indication in the subject and optionally do not provide a non-specific response; and adding a portion of the sample from the subject to each of the pools of (a) to determine if the sample generates the response.

Also disclosed is a computer-program product tangibly embodied in a non-transitory machine-readable storage medium including instructions configured to run a system comprising: a component or station for screening a plurality of biological samples for a response indicative of the presence of a predetermined indication; and a component or station for selecting a plurality of the samples that provide the response above a first predetermined threshold to generate the pool. In certain embodiments, the computer-program product further comprises instructions configured to run a system comprising a component or station to use the pool to test for a response indicative of the presence of the predetermined indication in a sample from a subject.

The systems and computer products may perform any of the methods disclosed herein. One or more embodiments described herein can be implemented using programmatic modules, engines, or components. A programmatic module, engine, or component can include a program, a sub-routine, a portion of a program, a software component, or a hardware component capable of performing one or more stated tasks or functions. As used herein, a module or component can exist on a hardware component independently of other modules or components. Alternatively, a module or component can be a shared element or process of other modules, programs, or machines.

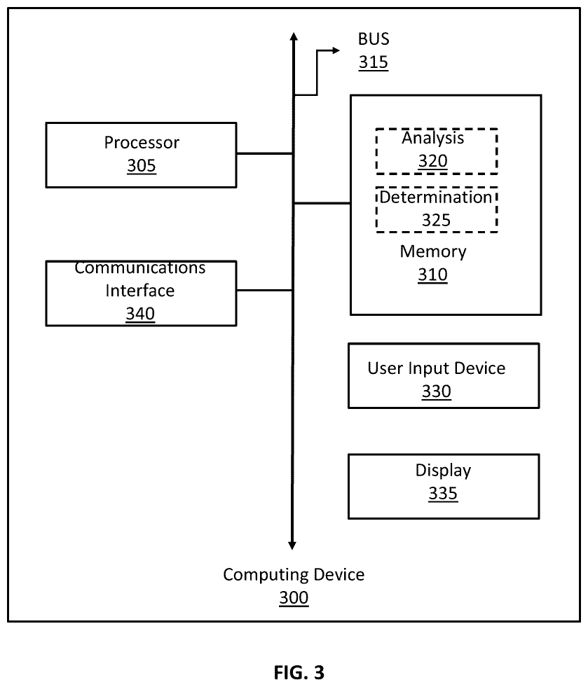

FIG. 3 shows a block diagram of an analysis system 300 used for detection and/or quantification of a progesterone metabolite. As illustrated in FIG. 3, modules, engines, or components (e.g., program, code, or instructions) executable by one or more processors may be used to implement the various subsystems of an analyzer system according to various embodiments. The modules, engines, or components may be stored on a non-transitory computer medium. As needed, one or more of the modules, engines, or components may be loaded into system memory (e.g., RAM) and executed by one or more processors of the analyzer system. In the example depicted in FIG. 3, modules, engines, or components are shown for implementing the methods or running any of the systems of the disclosure.

Thus, FIG. 3 illustrates an example computing device 300 suitable for use with systems and the methods according to this disclosure. The example computing device 300 includes a processor 305 which is in communication with the memory 310 and other components of the computing device 300 using one or more communications buses 315. The processor 305 is configured to execute processor-executable instructions stored in the memory 310 to perform one or more methods or operate one or more stations for detecting progesterone metabolite levels according to different examples, such as those in FIGS. 1-2 and 4-14 or disclosed elsewhere herein. In this example, the memory 310 may store processor-executable instructions 325 that can analyze 320 results for sample as discussed herein.

The computing device 300 in this example may also include one or more user input devices 330, such as a keyboard, mouse, touchscreen, microphone, etc., to accept user input. The computing device 300 may also include a display 335 to provide visual output to a user such as a user interface. The computing device 300 may also include a communications interface 340. In some examples, the communications interface 340 may enable communications using one or more networks, including a local area network (“LAN”); wide area network (“WAN”), such as the Internet; metropolitan area network (“MAN”); point-to-point or peer-to-peer connection; etc. Communication with other devices may be accomplished using any suitable networking protocol. For example, one suitable networking protocol may include the Internet Protocol (“IP”), Transmission Control Protocol (“TCP”), User Datagram Protocol (“UDP”), or combinations thereof, such as TCP/IP or UDP/IP.

The computer-usable or computer-readable medium may be, but is not limited to, an electronic, magnetic, optical, electromagnetic, infrared, or semiconductor system, apparatus, device, or propagation medium. More specific examples (a non-exhaustive list) of the computer-readable medium would include the following: an electrical connection having one or more wires, a portable computer diskette, a random-access memory (RAM), a read-only memory (ROM), an erasable programmable read-only memory (EPROM or Flash memory), an optical fiber, and a portable compact disc read-only memory (CD-ROM). The computer-usable or computer-readable medium could even be paper or another suitable medium, upon which the program is printed, as the program can be electronically captured, via, for instance, optical scanning of the paper or other medium, then compiled, interpreted, or otherwise processed in a suitable manner if necessary, and then stored in a computer memory.

Computer program code for carrying out operations of the present disclosure may be written in an object-oriented programming language such as Java7, Smalltalk, Python, Labview, C++, or VisualBasic. However, the computer program code for carrying out operations of the present disclosure may also be written in conventional procedural programming languages, such as the “C” programming language or even assembly language. The program code may execute entirely on the user's computer, partly on the user's computer, as a stand-alone software package, partly on the user's computer and partly on a remote computer or entirely on the remote computer. In the latter scenario, the remote computer may be connected to the user's computer through a local area network (LAN) or a wide area network (WAN), or the connection may be made to an external computer (for example, through the Internet using an Internet Service Provider).

### EXAMPLES

### Example 1

Test Principle: The basophil activation test (“BAT”) is a flow cytometry based functional assay that stimulates live cells in fresh whole blood and assesses IgE cross-linking and is a more precise allergic readout than measuring the concentration of allergen-specific IgE (Iqbal K, Bhargava K, Skov P S, Falkencrone S, Grattan C E. A Positive Serum Basophil Histamine Release Assay Is a Marker for Ciclosporin-Responsiveness in Patients With Chronic Spontaneous Urticaria. Clin Transl Allergy (2012) 2:19. doi: 10.1186/2045-7022-2-19; Santos A F, Douiri A, Becares N, et al. Basophil activation test discriminates between allergy and tolerance in peanut-sensitized children. J Allergy Clin Immunol. 2014; 134 (3): 645-652). The basophil activation test for chronic urticaria evaluates basophil activation in a whole blood pool upon patient serum (allergen) stimulation. The BAT assay uses the stain, lyse, wash method and a two antibody panel. The CCR3 (eosinophil and basophil marker) antibody used in this flow cytometry based assay selects for basophils, while CD63 (activation marker) is used to determine basophil degranulation. However, traditional methods for the basophil activation test require three readily available basophil donors who have high activation of anti-FcεRI which can be challenging for both the clinical testing facility and the blood donors. The present study defines an approach of pooling basophil donors from CBC routine testing to provide a consistent and reliable clinically validated assay for commercial laboratories. This CU-assay takes 2 days to complete. On Day 1, 32 or more basophil donors (ELISA Basophil IgG>0.2; i.e., 0.2 and 0.3, adding 0.1 if needed) were screened for basophil activation using the activation trigger anti-FcεRI mAb. The following day, the non-responders were eliminated and the 20 highest CD63+ responders to the anti-FcεRI mAb were selected, whose background was <4%, and whose response to the low-serum pool was also <4%. It was found that a background control is not required as the results with the low-serum pool were sufficient.

CSU Patient sera were then screened with the pooled blood. The precision of the assay was determined with low, intermediate, and high pooled serum. Furthermore, a technical cutoff of the pooled blood was defined for the assay with the central 95% of 142 normal serum patients with Total IgE (<80) using the Chauvenet method for outlier elimination.

### Definitions

CSU=Chronic Spontaneous Urticaria
BAT=Basophil Activation Test
ASST=Autologous Serum Skin Test
IgE=Immunoglobulin E
CD63=Cluster of Differentiation 63
CCR3=CC motif Receptor 3
RBC=Red Blood Cell
FSC=Forward Scatter
SSC=Side Scatter
FDA=Food and Drug Administration
SOP=Standard operating procedure

### Reagents:

Stimulation Buffer
Staining Reagent (antibody cocktail)
Anti-FcεRI mAb Stimulation Control
Wash Buffer
Lysing Reagent

In-house Low Serum Pool Control (LSP): To make the in-house Low Serum Pool Control (LSP), previously screened and stored serum with <4.67% activated basophils were pooled. The individual serum pooled for the Low Control resulted at 3 standard deviations below the technical cutoff of 10.61. All volume of the individual serums were combined in a 250 mL sterile bottle. Sub-aliquots of 5 mL and 1.2 mL were made and stored at −20° C.

In-house Intermediate Low Serum Pool Control (ISP): To make the in-house Intermediate Low Serum Pool Control (ISP), previously screened and stored serum between 4.67-8.63% activated basophils are pooled. The individual serum pooled for the intermediate low controls resulted between 1 and 3 standard deviation below the technical cutoff of 10.61. All volume of the individual serums were combined in a 250 mL sterile bottle. Sub-aliquots of 5 mL and 1.95 mL were made and stored at −20° C.

In-house High Serum Pool Control (HSP): To make the in-house High Serum Pool Control (HSP), previously screened and stored serum with >16.55% activated basophils are pooled. The individual serum pooled for the High Serum Control resulted greater than 3 standard deviations above the reference interval. All volume of the individual serums were combined in a 250 mL sterile bottle. Sub-aliquots of 5.0 mL and 250 μL were made and stored at −20° C. and sub-aliquots are made.

Parallel Testing: In order to maintain accuracy of laboratory reporting when a new lot number and/or shipment of reagents is utilized for patient testing, a parallel reagent check (lot-to-lot comparison) can be performed and documented prior to reporting patient results with the new lot and/or shipment. This involves testing the same patient samples and/or quality control (QC) material with reagents from the current and the new lots and/or shipments in order to compare results for similarity.

Quality Control: Low Serum Pool, Intermediate-Low Serum Pool and High Serum Pool controls were run with every batch of samples to ensure that all reagents and procedures perform properly. Expression values must be within the lot's expected results parameter.

### Sample Processing Protocol for Day 1:

Patient samples can be prepared manually, as is described in detail herein. Alternatively, patient samples can be processed via automated liquid handling. The working lysing reagent, working low serum pool lot and a 420 μL aliquot of the FcεRI stimulation control were brought to room temperature. Next the stimulation buffer was prepared (either by thawing frozen aliquots or reconstitution of lyophilized buffer with nuclease free water. Next the samples were prepared. Each basophil donor has 2 tubes (or 3 tubes if a background control is used). The selected basophil donors were then collected using samples from Lavender EDTA collection tubes, which have been collected the day before and have a basophil absolute count 0.20×10E3/μL-0.3×10E3/μL. The basophil donor whole blood was then mixed by inversion and 50 μL of patient whole blood added to the corresponding tubes, dispensing the volume at the bottom of the tube and not on the wall of the tube and covering the tubes with aluminum foil or parafilm.

At this point, the master mixes for the controls are created.

For the FcεRI stimulation control, 3.675 mL of stimulation buffer was transferred to an appropriately sized conical tube, 0.875 mL of the FcεRI stimulation control was added, and 700 μL of staining reagent was added. The tube was sealed and covered to avoid light exposure. The amount used for each FcεRI stimulation test was 105 μL of stimulation buffer, 25 μL FcεRI stimulation control, 20 μL staining reagent.

For the low serum pool in-house control, 2.8 mL of stimulation buffer was transferred to an appropriately sized conical tube, 1.75 mL of the low serum pool in-house control and the 700 μL of staining reagent was added. The tube was sealed and covered to avoid light exposure. The amount used for each FcεRI stimulation test was 80 μL of stimulation buffer, 50 μL low serum pool in-house control, 20 μL staining reagent.

At this point, 150 μL of the corresponding master mix was added to the tubes. The tubes were briefly vortexed to mix, covered with aluminum, and placed in the 37° C. water bath for 20 minutes. After incubation, 1.5 mL of lysis reagent was added to each tube and the tubes incubated at room temperature for 10 minutes. After gently vortexing, the tubes were centrifuged at 500×g for 5 min at room temperature with the break set at 2. The supernatant was then decanted, and the tubes blotted on absorbent pads. Next 300 μL of cold wash buffer was added to each tube, and the tube vortexed gently to re-suspend the cell pellet.

If acquisition could not be done right away, the tubes were incubated in the dark at room temperature for 30 minutes and then were stored overnight at 2-8° C. if the assay of a patient sample is to be performed the next day.

### Sample Acquisition:

During sample acquisition, it was confirmed that cells are collected in the SSC Singlet plot. At least 500 basophil events were acquired per tube. The acquisition time may vary from donor to donor.

It was also confirmed that for each processed sample 500 basophils were acquired. For any sample that didn't acquire, if the sample volume wasn't exhausted, an attempt was made to reacquire the tube. If sample volume is exhausted, that specimen will have to be re-processed. If a donor did not have at least 300 basophils events after a 200 second acquisition, the donor was excluded from pool selection.

### Gating and Batch Analysis:

After the batch acquisition is complete, the Chronic Urticaria Report is assessed for every donor to ensure each is gated properly and not including cells from neighboring population clusters.

The FcεRI control tube was selected as all gating was done in the positive control tube. On the report worksheet the Time dot-plot was adjusted to make sure to tighten the SSC singlets gate to discriminate any doublets and to make sure that the lower left corner (50 SSC-H×50 SSC-A area) was not gated out as the cells of interest may lie in this area. On the SSC Singlets plot, it was affirmed that the Total Leukocytes gate did not exclude the lymphocyte and monocyte region.

The gating on the Basophil plot was adjusted using the contour plot, along with the histogram to aid the placement of the gate. On the histogram plot, placed the Act. The basophils gate was placed on the positive peak followed by adjusting the quadrant gate on the same CD63-A location. The placement of the quadrant gate was close to the third decade.

### Selection of Basophil Donors for Whole Blood Pool

After gating is complete, 20 donors were selected for day 2 procedures (i.e., assay of patient samples). Donors who were non-responders to the anti-FcεRI stimulation (i.e., <10% activated basophils for the FcεRI control) were excluded. Additionally, donors were excluded if they had innate basophil activation reflected as a high percentage of activated basophils in the optional background and/or LSP control. Donors were also excluded if in the SSC singlets plot one of the following populations is missing: lymphocytes, monocytes, granulocytes. Donors were also excluded if they did not have at least 300 basophil events within 200 seconds of acquisition.

Table 1 is an example of basophil donor screening.

TABLE 1

| | Basophil | FcεRI % | LSP % | |
|---|---|---|---|---|
| Donor | Absolute | Activated | Activated | |
| # | Count | Basophil | Basophil | Notes |
| 1 | 0.2 | 40.77 | 0.37 | |
| 2 | 0.2 | 0.99 | 0.17 | Non-responder |
| 3 | 0.2 | 71.8 | 0.39 | Selected for 20 donor pool |
| 4 | 0.2 | 91.31 | 0.98 | Selected for 20 donor pool |
| 5 | 0.2 | 94.01 | 2.66 | Selected for 20 donor pool |
| 6 | 0.2 | 92.65 | 0.37 | Selected for 20 donor pool |
| 7 | 0.2 | 90.93 | 0.58 | Selected for 20 donor pool |
| 8 | 0.2 | 33.14 | 1.37 | |
| 9 | 0.2 | 15.02 | 0 | |
| 10 | 0.2 | 89.24 | 0.16 | Selected for 20 donor pool |
| 11 | 0.2 | 94.79 | 3.51 | |
| 12 | 0.2 | 53.89 | 0.3 | |
| 13 | 0.2 | 88.95 | 0.49 | Selected for 20 donor pool |
| 14 | 0.2 | 79.53 | 0.94 | Selected for 20 donor pool |
| 15 | 0.2 | 81.61 | 0.75 | Selected for 20 donor pool |
| 16 | 0.2 | 93.01 | 1.63 | Selected for 20 donor pool |
| 17 | 0.2 | 91.7 | 2.27 | Selected for 20 donor pool |
| 18 | 0.2 | 92.3 | 0.8 | Selected for 20 donor pool |
| 19 | 0.2 | 92.23 | 1.66 | Selected for 20 donor pool |
| 20 | 0.2 | 90.74 | 79.46 | Excluded, high LSP stimulation |
| 21 | 0.2 | 12 | 0.47 | |
| 22 | 0.2 | 69.33 | 0.77 | Selected for 20 donor pool |
| 23 | 0.2 | 82.22 | 1.1 | Selected for 20 donor pool |
| 24 | 0.2 | 93.6 | 0.3 | Selected for 20 donor pool |
| 25 | 0.2 | 55.8 | 0.4 | |
| 26 | 0.2 | 58.8 | 0.5 | Selected for 20 donor pool |
| 27 | 0.2 | 3.65 | 0.49 | Non-responder |
| 28 | 0.2 | 96.96 | 1.53 | Selected for 20 donor pool |
| 29 | 0.2 | 89.87 | 0.33 | Selected for 20 donor pool |
| 30 | 0.2 | 90.19 | 0.2 | Selected for 20 donor pool |
| 31 | 0.2 | 93.7 | 2.9 | Selected for 20 donor pool |
| 32 | 0.2 | 96.58 | 1.03 | Selected for 20 donor pool |

### Sample Processing Protocol for Day 2

Patient specimens can be processed manually, as is disclosed in detail herein. Alternatively, patient specimens can be processed via automated liquid handling.

All patient serum, reagents, and controls (LSP, ISP, and HSP; FcεRI stimulation control) were brought to room temperature and the following steps performed.

Label a batch set of 12×75 mm tubes as follows:
Tube/Well 1: Background Control.
Tube/Well 2: FcεRI stimulation control
Tube/Well 3: Low serum pool
Tube/Well 4: Intermediate low serum pool
Tube/Well 5: High serum pool.
Tube/Well 6-XX: Patient serum

The selected basophil donors were removed from the refrigerator at least 5 minutes prior to pooling the donors and the tubes inverted (e.g., 5-7 times) to mix. The basophil donors to pool were inspected verify that the hemolysis was not present in the selected donors. If hemolysis was present, the next best donor was selected that fit the criteria to include in the pool.

Added 50 μL of patient serum to the corresponding tubes (tube/well #6 and after).

Whole blood from each selected donor (about 0.5-1.0 mL) was transferred into a labeled basophil pool conical tube, inverting each donor tube to mix the sample before transferring. After pooling blood from all 20 donors, the tube was inverted to mix. Next, the calculated whole blood pool volume was transferred into the master mix conical tube and pipetted to mix. Next, the calculated staining reagent (antibody) volume was transferred and pipetted to mix. Next, 150 μL of the pooled donor master mix was transferred to each tube, and the tubes briefly vortexed to mix.

The tubes were then transferred to 37° C. water bath for 20 minutes. After the water bath incubation, 1.5 mL of the lysis solution was added and the tubes incubated in the dark at room temperature for 10 minutes. The tubes were then gently vortexed for 1 second before centrifuging for 5 minutes at 500×g, room temperature, break set at 2. After decanting the supernatant, 300 μL of cold wash buffer was added to each tube and the tubes vortexed gently to re-suspend the cell pellet.

If acquisition was not done right away, the tubes were incubated in the dark at room temperature for 30 minutes and then stored at in 2-8° C. for up to 72 hours.

### Sample Acquisition

During sample acquisition, it was confirmed that cells are collected in the SSC Singlet plot, 1000 basophil events are to be acquired per tube.

It was also confirmed that for each processed sample, 1000 basophils were acquired. For any sample that didn't acquire, if the sample volume wasn't exhausted, it was attempted to reacquire the tube. If sample volume was exhausted, that specimen was re-processed.

### Day 2 Gating and Batch Analysis

Gating was the same as day 1 for “all events,” time, SSC singlets, and total leukocytes plots.

To gate for the BASOPHILS plot, the HSP control and the anti-FcεRI control tubes were used. The high serum pool (HSP) control had both distinctive positive and negative CD63 populations, whereas the anti-FcεRI control may have a clearly defined positive population and the negative population may not be as defined.

Selected the HSP control tube/well. Adjusted the ‘Act. Basophils’ gate on the histogram plot to gate CD63+, then clicked on the anti-FcεRI control to verify the histogram gating. The gating is typically close to the 3rd decade (103 notch).

On the anti-FcεRI control tube, adjusted the gating on the Basophil scatterplot to match the Basophils histogram as closely as possible. Attention was given to the location of the histogram gate (the decade number and the notches) and adjust the gate on the scatterplot.

To verify that the gating placement on the scatterplot is close to the histogram gate, compared the activated basophils and the Act. Basophils percent parent (% Parent) value on the run pointer statistics.

### Reporting

For the chronic uricaria BAT-Negative result: <10.61%; Positive result: ≥10.61%.

### Example 2

### Method

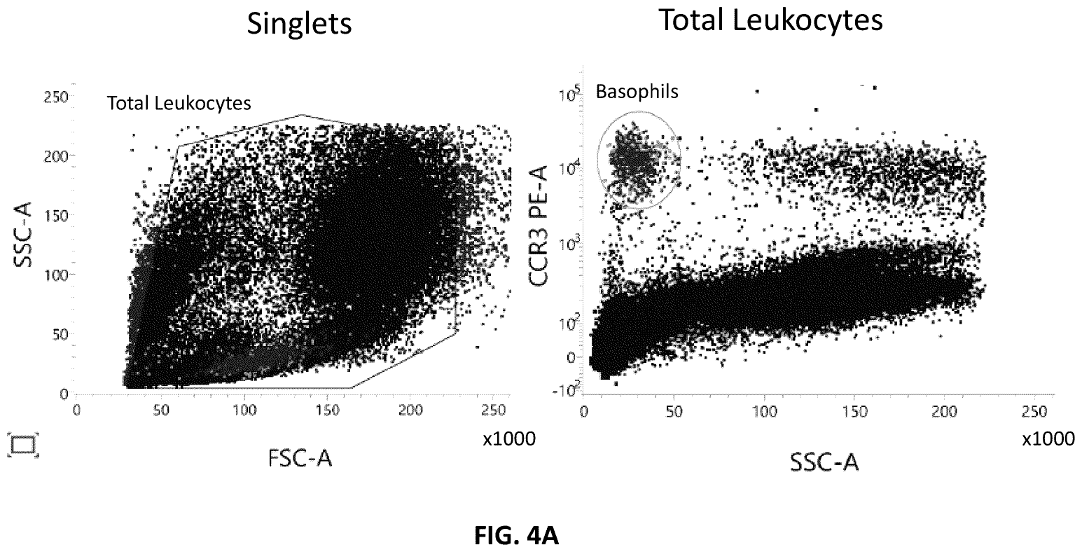

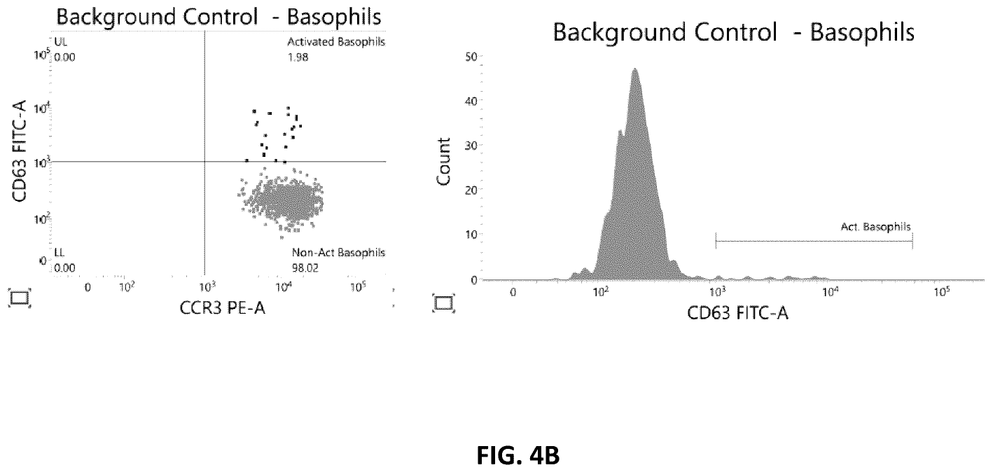

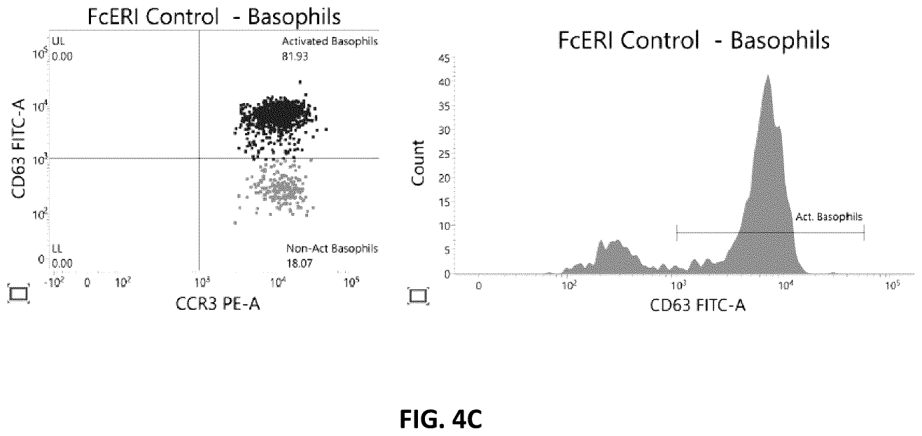

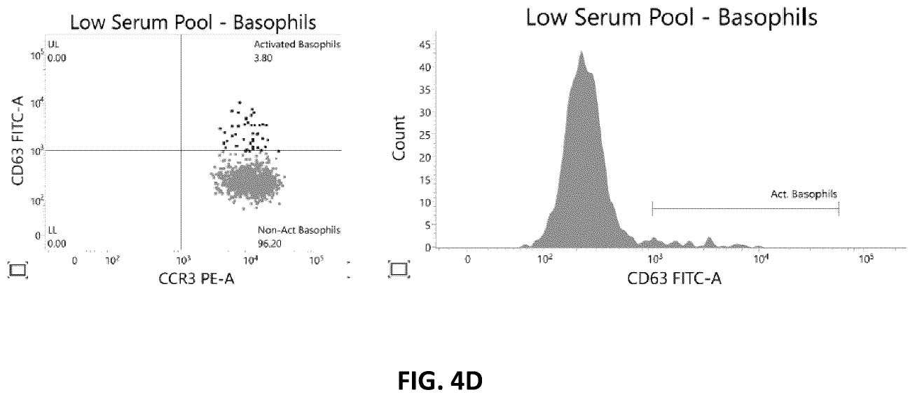

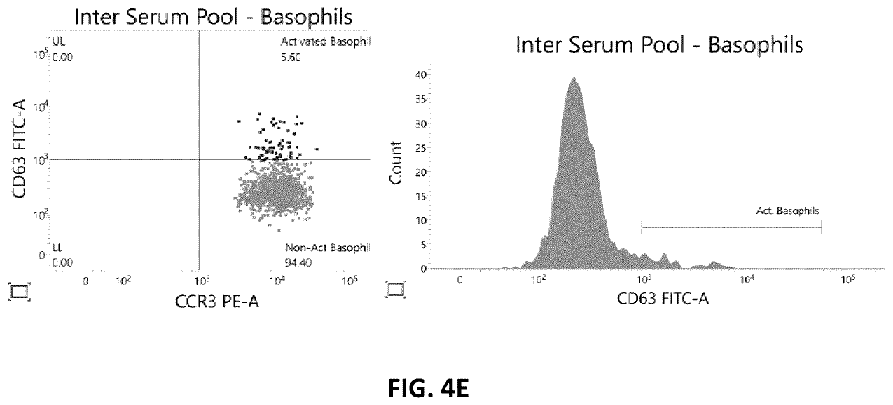

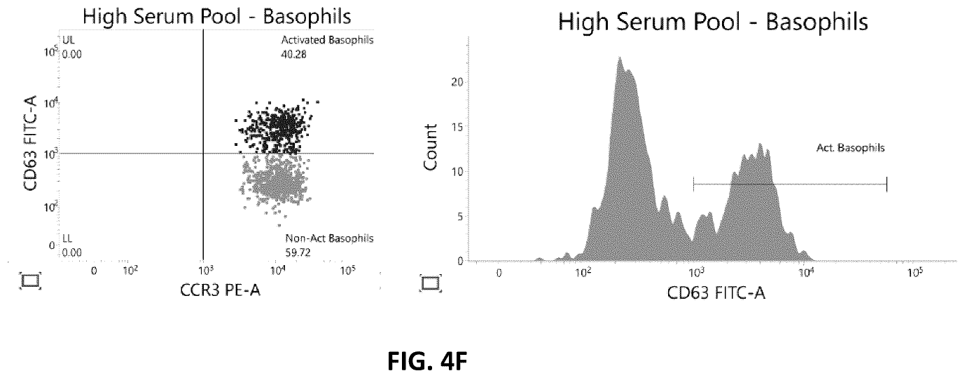

30 basophil donors (ELISA Basophil IgG>0.2) were screened for basophil activation using the activation trigger anti-FcεRI mAb. The following day, the 20 highest CD63+ responders were selected and the non-responders were eliminated, and a pool of 20 blood donors was prepared. CSU Patient sera were then screened with the pooled blood. Representative flow cytometry gating of a background control, an anti-FcεRI control, a low serum pool, an intermediate serum pool, and a high serum pool are shown in FIGS. 4A-4F. The precision of the assay was determined with low, intermediate, and high pooled serum. Furthermore, a technical cutoff of the pooled blood was defined for the assay with the 95% CI of 120 normal serum patients with Total IgE (<80) using the Chauvenet method for outlier elimination.

### Results

Precision was demonstrated for 10 unique basophil donor pools for 10 days. The background for pooled basophils was set to 3.8%-4.0% based on the natural separation between activated and non-activated basophils for multiple donor pools. For the 10 days, the average of the pools activated by the anti-FcεRI mAb 86.3% (4.3% CV) across the 10 experiments with precision demonstrated for the low, intermediate and high pooled serum.

### CONCLUSION

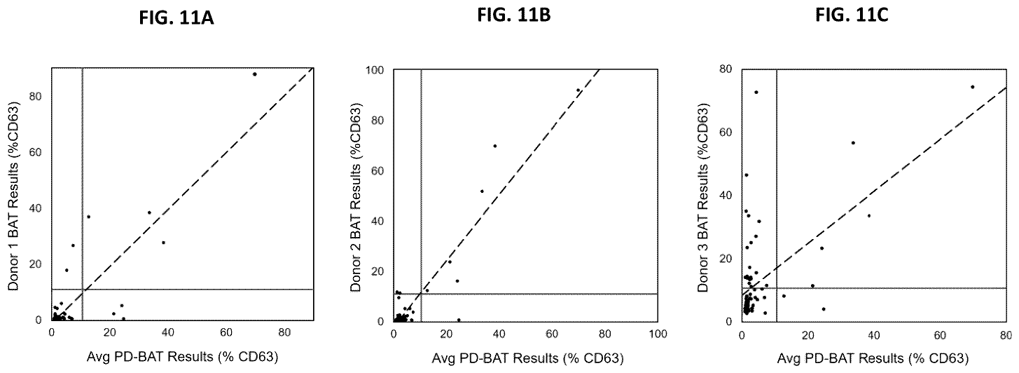

BAT is seen as an in vitro surrogate of patient allergic reactions and thus supports the diagnosis of CSU. This example demonstrates a robust laboratory method using pooled basophil donors for the expansion of BAT testing in commercial laboratories. A comparison of the pooled donor method disclosed herein and an individual donor method is described in more detail in Example 3 and shown in FIGS. 11A, 11B and 11C. The standardization of this assay with results that have been clinically validated tremendously advances patient diagnostics for CSU.

### Example 3

### Materials and Methods

### Basophil Activation by Stimuli or Patient Serum

The CD63 abundance on the surface of donor basophils was determined by flow cytometry. Flow CAST® (BUHLMANN Laboratories AG) was adapted for use in CSU patients as previously described (Gentinetta et al., 2011). Briefly, 50 μL of donor blood was mixed with 50 μL of patients' serum (or other stimuli), 100 μL of stimulation buffer (which contains IL-3), and 20 μL of antibody mix (contains fluorescein isothiocyanaten-labeled anti-CD63 antibody and phycoerythrin-labeled anti-CCR3 antibody). Basophil activation and staining was achieved by incubation for 20 min at 37° C. Anti-FcεRI antibody (50 μL) served as positive control and 50 μL of stimulation buffer served as background control. Red blood cells were lysed for 10 minutes in the dark at ambient temperature. The samples were centrifuged (500×g, 5 min, ambient temperature, low break setting), decanted and the pellets were re-suspended in 300 μL of the cold wash buffer containing 0.1% formaldehyde for cell fixing. If sample acquisition was not completed immediately after processing, the samples were incubated for 30 minutes at room temperature in the dark to fix cells, before storing at 2-8° C. up to maximum 3 days in the dark.

### Flow Cytometry Acquisition and Analysis

Basophils were identified by their CCR3 expression. At least 1000 basophil events were acquired for basophil screening. For the analysis, a time plot was used to observe the sample flow for all the events. A side scatter singlet plot was created from all the events, to discriminate doublets. From the side scatter singlets, a gate is applied to the total leukocytes. From the total leukocyte gate, CCR3+ basophils were identified. Following the basophils gate, a dual fluorochrome gate was used to analyze the activation of basophils (CCR3+CD63+). For the analysis of the basophil donor screening (day 1), the anti-FcεRI stimulation control for each donor was used for gating. For the analysis of patient serum screening (day 2), the minima between activated and non-activated basophil population using anti-FcεRI stimulation control was used to establish the gating, the High Serum Pool (HSP) control was used to make any minimal adjustments to the CD63+ population. Data analysis of the flow cytometry screening results was performed with R, version 4.2.5 (R Core Team), using package ggplot2 (Wickham 2016).

### Selection of Basophil Donors and Pool Production

De-identified EDTA whole blood samples submitted to a large reference laboratory for complete blood counts (CBC) with basophil absolute count ranging from 0.2 to 0.3×10E3/μL were selected within 24 hours of collection. The reactivity of the donor cells was tested by incubation with 1) stimulation buffer (background), 2) anti-FcεRI mAb and 3) our Low Serum Pool (LSP); see section 2.5 for details on pool production specifications. Donors showing high baseline activation (>4%) by the background control, non-specific activation (>4%) by the LSP or low response (<40%) to the anti-FcεRI control were excluded from the donor pool.

Additionally, donors were excluded if any of the following populations were missing in the SSC singlets plot: lymphocytes, monocytes, or granulocytes. Basophil donors were excluded if the basophil population was not clearly distinguishable between SSC and CCR3, to prevent inadequate gating and results. Thirty or more potential donors were screened for each testing batch and the 20 best performing donors (highest anti-FcεRI mAb response with other criteria met) were pooled and employed for the PD-BAT testing of patient sera.

### Patient Serum Testing by PD-BAT

For each batch of testing, the controls (background, anti-FcεRI stimulation and low (LSP), intermediate (ISP), and high serum pools (HSP) were processed along patient serum samples. Batch testing was initiated within 10 minutes of donor pool production. If flow cytometry acquisition was not completed after processing, the samples were incubated in the wash buffer containing 0.1% formaldehyde for 30 minutes at room temperature in the dark to fix cells and stored at 2-8° C. for up to 3 days. At least 1000 basophil events were acquired for serum testing.

### Preparation of Serum Pool Controls

Sera that tested ≥3.0 standard deviations (SD) above the PD-BAT technical cut-off were pooled for the high serum pool (HSP). Sera that tested >3 SD below the technical cut-off were pooled to create the low serum pool (LSP). Sera that tested between 1 to 3 SD below the technical cut-off were pooled to form the intermediate serum pool (ISP). Sub-aliquots of these serum control pools were then stored at −20° C. for future use.

### Reproducibility and Precision

PD-BAT measurements were performed in quadruplicate for the background, anti-FcεRI, LSP, ISP, and HSP. These internal control serum pools were stored as frozen aliquots prior to re-equilibrating to room temperature for daily testing. PD-BAT measurements were performed in quadruplicate on ten separate days. Intra- and inter-assay reproducibility were calculated for the five materials.

### Pooled Whole Blood Stability for the PD-BAT

The background, anti-FcεRI control, LSP, ISP, HSP were tested at baseline, 2—, 4-, 6-, and 24-hours post-donor pool production to evaluate the stability of the pooled whole blood. The pooled whole blood was stored at 2-8° C. between time points.

### Processed Samples Stability Analysis for PD-BAT

The controls (background, anti-FcεRI control, LSP, ISP, HSP) and serum samples were processed for acquisition at baseline and every 24 hours post-processing for up to 120 hours. The processed samples were stored in the dark at 2-8° C.

### Serum Temperature Stability for PD-BAT

The stability of results produced from serum samples stored under three temperature conditions (ambient, refrigerated, and frozen) was assessed using the LSP, ISP, HSP for PD-BAT. For the ambient and refrigerated (2-8° C.) conditions, isochronic studies were conducted with the serum pools for 14, 7, 3, 1 and 0 days (baseline). For the frozen stability study, the serum pools were tested at the baseline and at intervals up to 71 days.

### Reagent and Key Component Stability Study

The performance of the staining reagent stored under dark ambient conditions was assessed for up to 3 days. The controls (background, anti-FcεRI control, ISP, LSP, HSP) and 19 serum samples were tested with staining reagent for each staining reagent condition and time point. The anti-FcεRI stimulation control was reconstituted and refrigerated for 1 and 7 days and compared to the baseline for its stability.

### PD-BAT Reference Interval and Technical Cut-Off Determination

A total of 144 serum samples with total IgE (ThermoFisher ImmunoCAP™) levels below 80 IU/mL were selected for technical cut-off determination to reduce the likelihood of including atopic individuals in the reference population (Chang et al., Analysis of total immunoglobulin E and specific immunoglobulin E of 3,721 patients with allergic disease, Biomed Rep., 2015 July; 3 (4): 573-577. doi: 10.3892/br.2015.455. Epub 2015 Apr. 29. PMID: 26171168; PMCID: PMC4486958). Reference intervals were generated by transformed-parametric analysis using EP Evaluator® (Data Innovations, Burlington, Vermont, USA) following removal of outliers using Chauvenet's criteria. The clinical cut-off was verified with an additional 157 specimens that tested below the lower limit of detection of a basophil histamine release assay (CU Index®, Eurofins Viracor, Lenexa, KS).

BAT Testing with Individual Donors

The Flow CAST® protocol according to Instruction for Use (IFU) was performed for screening healthy basophil donors. Selection criteria for three basophil donors included: non-responsiveness to anti-FcεRI (<10% CD63 activation), high anti-FcεRI response (>65% CD63 activation), high basophil count (>1000 cells per 50 μL whole blood), and a low basophil activation to a Negative Serum Pool (NSP, <10% CD63 activation). The negative serum pool, similar in purpose to the LSP (Low Serum Pool), comprised serum from ostensibly healthy individuals, serving to exclude donors with spontaneous activation. In addition, the leukocyte population should be separated into three discrete populations (lymphocytes, monocytes and granulocytes) on the FSC/SSC dot plot.

### Method Comparison of Individual Donor CSU-BAT Versus PD-BAT

A set of 69 CSU patient samples was analyzed with BAT testing performed by the traditional method (with three individual donors) and by the PD-BAT method. BUHLMANN Laboratories AG conducted the traditional method testing and Labcorp conducted the PD-BAT testing.

### BHRA Comparison to PD-BAT

343 samples tested by CU-Index® were also tested by the PD-BAT assay. Results were compared using EP Evaluator Statistical Models (Qualitative Method Comparison, Qualitative Method Comparison).

### Results

### Donor Screening Results

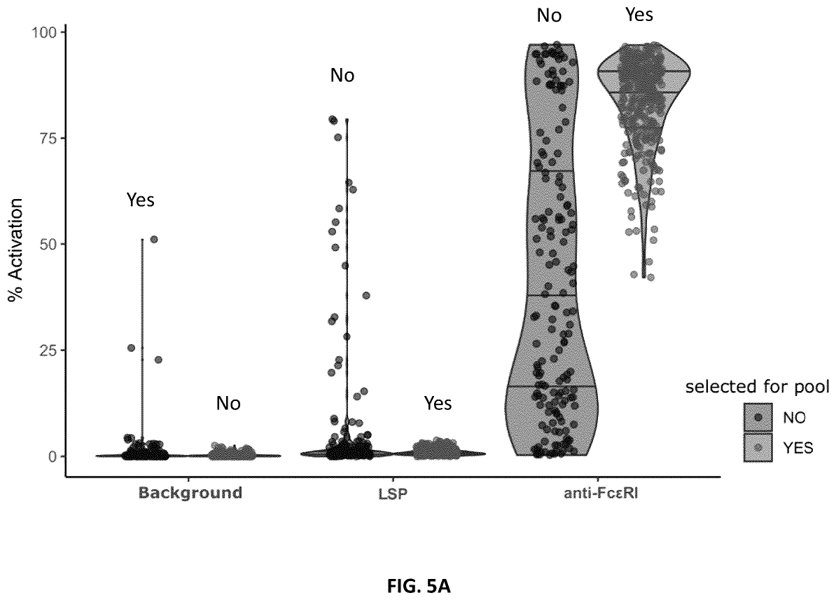

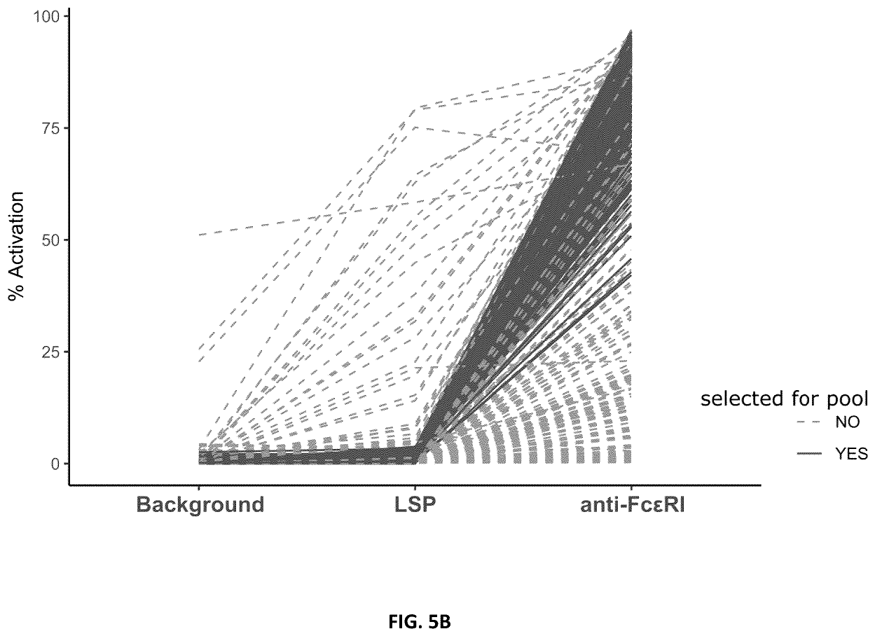

In the initial phase of the study, potential basophil donors were screened to ensure the robustness of our Pooled Donor Basophil Activation Test (PD-BAT). The screening process aimed to identify responsive donors while excluding those with heightened background activation. Results were based on the gating strategy using the positive control and HSP as described in the methods above. 1.1% of screened potential donors produced an activation with addition of stimulation buffer alone of >4% CD63+ (elevated background activation). 6.4% produced an activation to the low serum pool LSP>4% CD63+ (non-specific response). 7.1% failed to produce an activation to the positive control, anti-FcεRI of >10% CD63+ (non-responder). 18.5% failed to produce an activation to the positive control, anti-FcεRI, of >40% CD63+ (poor responder). As a result of the combination of criteria used, 13.7% of 463 screened donors were excluded from use in creating donor pools over all experiments (FIGS. 5A and 5B).

### Reproducibility and Precision

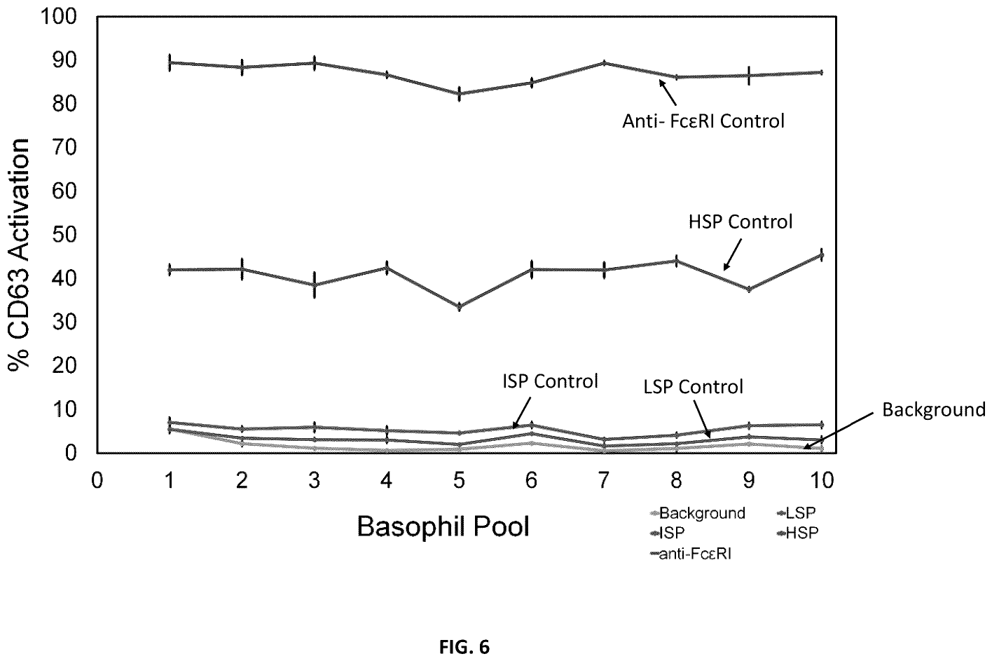

The evaluation of reproducibility and precision in the PD-BAT method is vital for its reliability in clinical applications. The results highlights remarkable intra-assay and inter-assay comparability as illustrated in FIG. 6 and summarized in Table 2. In Table 2, intra-assay results were calculated from as assay run with 10 replicates for each sample. The inter-assay results were derived from combining a replicate for each sample tested over 10 different pools. Specifically, the intra-assay coefficient of variation (CV) was low at 1.92% for anti-FcεRI, and 4.57% for HSP. Similarly, the inter-assay CV remained low at 3.44% for anti-FcεRI, and 9.56% for HSP. Notably, the CV for the Background, LSP, and ISP were expectedly higher due to their derivation from smaller numbers, which amplifies any changes. Furthermore, a 99% confidence limit (+/−3 standard deviations) was found for the positive control, and internal serum controls, further affirming the reliability of the findings.

TABLE 2
Inter and Intra-assay reproducibility of PD-BAT

| | Intra Assay | Intra Assay | Intra Assay | Inter Assay | Inter Assay | Inter Assay |
|---|---|---|---|---|---|---|
| Sample | Mean | % CV | SD | Mean | % CV | SD |
| Background | 0.87 | 34.11 | 1.79 | 1.77 | 101.20 | 1.48 |
| LSP | 1.95 | 18.33 | 1.38 | 3.39 | 40.66 | 1.19 |
| ISP | 4.57 | 14.57 | 1.38 | 5.88 | 23.48 | 1.36 |
| HSP | 34.42 | 4.57 | 3.90 | 40.78 | 9.56 | 3.67 |
| anti-FcεRI | 83.43 | 1.92 | 3.00 | 87.28 | 3.44 | 2.48 |

### Serum Stability

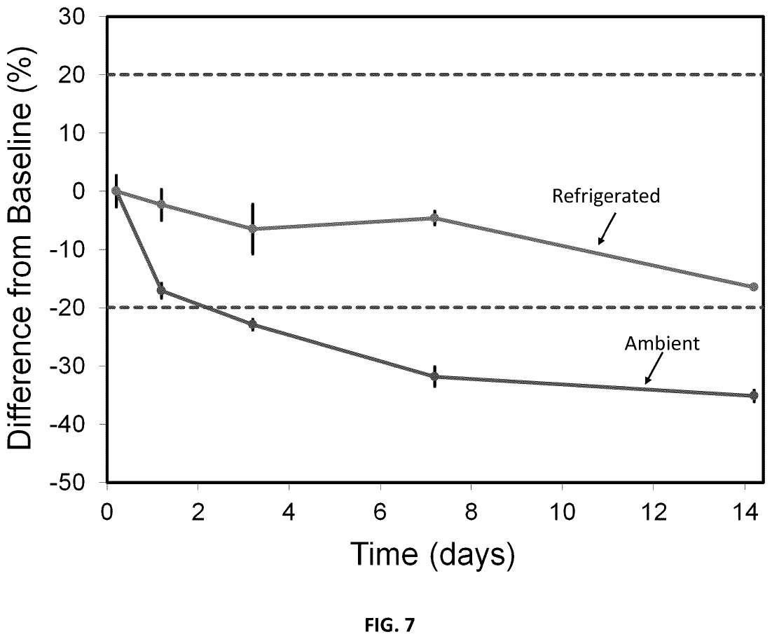

The stability of the high serum pool (HSP) at baseline and at intervals up to 14 days with room temperature (20-23° C.) and refrigerated (4-8° C.) storage was assessed (FIG. 7). Recovery of the room temperature stored sampled dropped 17.1% within 24 hours and more than 20% with longer storage. The refrigerated sample was stable (i.e., reduction <20%) for 14 days. Frozen stability was demonstrated in the inter-assay precision study (FIG. 6) where results did not drop over the course of an experiment where ten assays were performed on controls and pools tested over the course of 71 days. These findings indicate the importance of timely testing of serum for the successful clinical application of the PD-BAT.

### Pooled Whole Blood Donor Stability

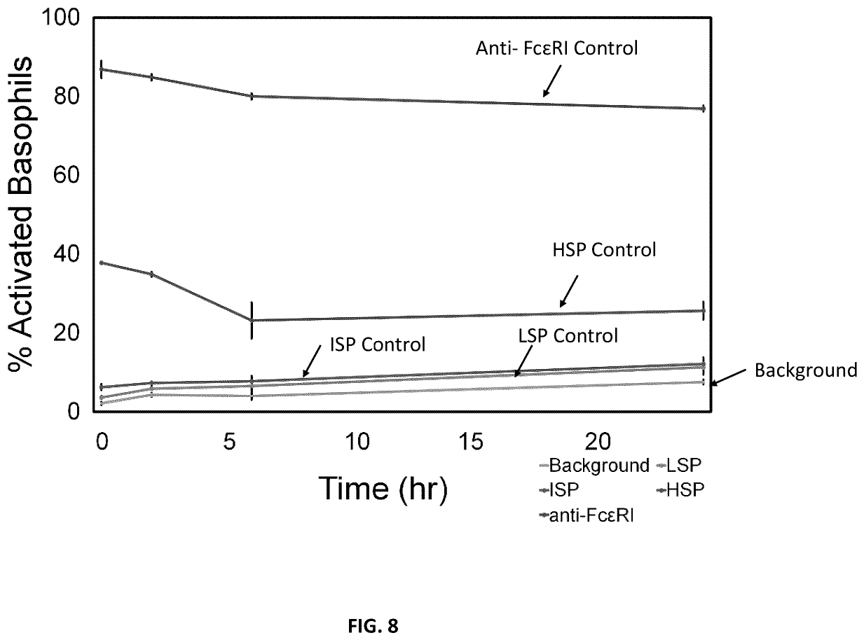

A whole blood pool was produced and used to test serum pools and controls by PD-BAT at a number of time points up to 24 hours (FIG. 8). The recovery for the HSP dropped 8.5% in 2 hours and 35.7% in 6 hours. The recovery of the LSP increased from 3.6% at baseline to 11.3% at 24 hours. These results indicate that the donor pool should be used for testing promptly after the preparation (within 2 hours).

### Processed Sample Stability Analysis

PD-BAT test samples that have been processed to the point of loading onto the flow cytometer remain stable for up to 72 hours when stored under refrigerated conditions (Table 3). In Table 3, the controls and 20 serum specimens were processed and analyzed at 0 (baseline), 24, 48, 72, 96, and 120 hours. The change in activated basophil read-out from the baseline was evaluated to determine the stability of the processed samples. Beyond the 72-hour mark, substantial variations were observed, particularly in the background of the samples, highlighting the importance of timely analysis after processing.

### Critical Reagent Stability

Critical reagents (anti-FcεRI control antibody, the staining reagent containing CCR3-PE and CD63-FITC) were reconstituted and used for testing (baseline). The anti-FcεRI control was evaluated after a 7-day refrigerated storage period. The staining reagent was assessed after a 3-day period at room temperature (20-23° C.) in the dark. The analysis revealed a consistent categorical agreement across the controls and sera tested under both conditions (Tables 4 and 5). In Table 4 The stability of the staining reagent was tested at the baseline and after a 3 day storage under dark and ambient conditions. For each timepoint the basophil activation for 5 controls and 20 sera were measured (n=1). In Table 5, the stability of the stimulation control was tested for the baseline and after 1 and 7 day storage at 2-8° C. For each timepoint the basophil activation for 5 controls and 20 sera were measured (n=1).

TABLE 3
Processed Sample Stability for PD-BAT

| | | | 24 hr Stability | | 48 hr Stability | | 72 hr Stability | | 96 hr Stability | | 120 hr Stability | |
|---|---|---|---|---|---|---|---|---|---|---|---|---|
| | | Baseline | | % Δ | | % Δ | | % Δ | | % Δ | | % Δ |
| | | % Acti- | % Acti- | from | % Acti- | from | % Acti- | from | % Acti- | from | % Acti- | from |
| | | vated | vated | Base- | vated | Base- | vated | Base- | vated | Base- | vated | Base- |
| | Specimen | Basohlis | Basohlis | line | Basohlis | line | Basohlis | line | Basohlis | line | Basohlis | line |
| Controls | Background | 1.76 | 1.77 | 0.57 | 2.24 | 27.27 | 3.22 | 82.95 | 13.61 | 673.30 | 40.59 | 2206.25 |
| | LSP | 3.34 | 3.22 | −3.59 | 3.60 | 7.78 | 3.99 | 19.46 | 3.88 | 16.17 | 2.62 | −21.56 |
| | ISP | 5.03 | 4.43 | −11.93 | 5.71 | 13.52 | 5.09 | 1.19 | 4.80 | −4.57 | 4.32 | −14.12 |
| | HSP | 33.14 | 35.28 | 6.46 | 35.07 | 5.82 | 39.17 | 18.20 | 33.95 | 2.44 | 37.36 | 12.73 |
| | anti-FcERI | 88.05 | 89.37 | 1.50 | 88.28 | 0.26 | 89.40 | 1.53 | 89.17 | 1.27 | 91.32 | 3.71 |
| Specimen | Serum 1 | 5.38 | 4.41 | −18.03 | 4.70 | −12.64 | 4.40 | −18.22 | 3.54 | −34.20 | 3.44 | −36.06 |
| | Serum 2 | 6.49 | 5.79 | −10.79 | 5.89 | −9.24 | 5.86 | −9.71 | 5.45 | −16.02 | 6.04 | −6.93 |
| | Serum 3 | 9.38 | 7.98 | −14.93 | 11.84 | 26.23 | 10.26 | 9.38 | 9.05 | −3.52 | 7.75 | −17.38 |
| | Serum 4 | 4.41 | 4.79 | 8.62 | 5.58 | 26.53 | 5.32 | 20.63 | 4.77 | 8.16 | 4.50 | 2.04 |
| | Serum 5 | 9.60 | 11.51 | 19.90 | 11.41 | 18.85 | 9.07 | −5.52 | 7.65 | −20.31 | 6.64 | −30.83 |
| | Serum 6 | 14.40 | 14.42 | 0.14 | 13.41 | −6.88 | 13.17 | −8.54 | 10.24 | −28.89 | 9.41 | −34.65 |
| | Serum 7 | 8.02 | 7.16 | −10.72 | 7.66 | −4.49 | 5.82 | −27.43 | 5.87 | −26.81 | 5.63 | −29.80 |
| | Serum 8 | 6.68 | 7.72 | 15.57 | 6.80 | 1.80 | 6.17 | −7.63 | 6.35 | −4.94 | 6.36 | −4.79 |
| | Serum 9 | 4.58 | 4.73 | 3.28 | 4.94 | 7.86 | 5.64 | 23.14 | 3.68 | −19.65 | 5.36 | 17.03 |
| | Serum 10 | 4.82 | 4.31 | −10.58 | 5.16 | 7.05 | 4.32 | −10.37 | 2.96 | −38.59 | 4.72 | −2.07 |
| | Serum 11 | 56.28 | 58.01 | 3.07 | 60.07 | 6.73 | 55.92 | −0.64 | 52.66 | −6.43 | 52.16 | −7.32 |
| | Serum 12 | 26.40 | 24.63 | −6.70 | 25.38 | −3.86 | 22.39 | −15.19 | 18.12 | −31.36 | 19.30 | −26.89 |
| | Serum 13 | 25.31 | 27.10 | 7.07 | 30.10 | 18.93 | 28.45 | 12.41 | 24.06 | −4.94 | 25.07 | −0.95 |
| | Serum 14 | 11.76 | 11.31 | −3.83 | 12.94 | 10.03 | 12.67 | 7.74 | 11.61 | −1.28 | 10.02 | −14.80 |
| | Serum 15 | 39.63 | 37.92 | −4.31 | 39.61 | −0.05 | 36.89 | −6.91 | 35.80 | −9.66 | 40.07 | 1.11 |
| | Serum 16 | 73.83 | 73.54 | −0.53 | 77.60 | 4.96 | 75.95 | 2.73 | 67.23 | −9.06 | 68.30 | −7.62 |
| | Serum 17 | 17.24 | 15.67 | −9.11 | 17.23 | −0.06 | 15.50 | −10.09 | 14.98 | −13.11 | 15.25 | −11.54 |
| | Serum 18 | 20.00 | 16.31 | −18.45 | 20.55 | 2.75 | 19.92 | −0.40 | 16.50 | −17.50 | 16.24 | −18.80 |
| | Serum 19 | 46.41 | 42.26 | −8.94 | 46.68 | 0.58 | 41.59 | −10.39 | 40.54 | −12.65 | 38.11 | −17.88 |
| | Serum 20 | 44.81 | 43.77 | −2.32 | 44.59 | −0.49 | 43.10 | −3.82 | 38.97 | −13.03 | 37.40 | −16.54 |

TABLE 4
Stability of Staining Reagent

| | | Baseline | 3 Day | % Δ from |
|---|---|---|---|---|
| | Specimen Name | (CD63%) | (CD63%) | Baseline |
| Controls | Background | 0.70 | 0.30 | −57.14 |
| | LSP | 3.20 | 2.90 | −9.38 |
| | ISP | 4.10 | 4.20 | 2.44 |
| | HSP | 38.31 | 39.11 | 2.09 |
| | anti- FcERI | 81.44 | 83.28 | 2.26 |
| Specimen | Serum 1 | 0.29 | 0.88 | 203.45 |
| | Serum 2 | 0.88 | 0.68 | −22.73 |
| | Serum 3 | 1.36 | 1.02 | −25.00 |
| | Serum 4 | 0.97 | 1.05 | 8.25 |
| | Serum 5 | 1.28 | 1.27 | −0.78 |
| | Serum 6 | 0.39 | 0.58 | 48.72 |
| | Serum 7 | 3.80 | 3.30 | −13.16 |
| | Serum 8 | 0.79 | 0.87 | 10.13 |
| | Serum 9 | 1.77 | 1.27 | −28.25 |
| | Serum 10 | 0.87 | 0.97 | 11.49 |
| | Serum 11 | 21.48 | 24.14 | 12.38 |
| | Serum 12 | 39.81 | 43.46 | 9.17 |
| | Serum 13 | 6.39 | 6.53 | 2.19 |
| | Serum 14 | 71.88 | 72.00 | 0.17 |
| | Serum 15 | 6.72 | 6.70 | −0.30 |
| | Serum 16 | 51.02 | 52.46 | 2.82 |
| | Serum 17 | 27.82 | 26.92 | −3.24 |
| | Serum 18 | 23.73 | 20.80 | −12.35 |
| | Serum 19 | 19.33 | 19.40 | 0.36 |
| | Serum 20 | 63.30 | 58.79 | −7.12 |

TABLE 5
Anti-FcεRI mAb reagent stability

| | | Baseline | 1 Day Stability | | 7 Day Stability | |
|---|---|---|---|---|---|---|
| | | % Activated | % Activated | % Δ from | % Activated | % Δ from |
| | Specimen | Basohils | Basohils | Baseline | Basohils | Baseline |
| Controls | Background | 3.12 | 2.90 | −7.05 | 2.88 | −7.69 |
| | LSP | 5.00 | 5.70 | 14.00 | 4.48 | −10.40 |
| | ISP | 6.94 | 7.00 | 0.86 | 7.09 | 2.16 |
| | HSP | 34.80 | 39.00 | 12.07 | 35.11 | 0.89 |
| | anti-FcERI | 80.08 | 86.50 | 8.02 | 86.24 | 7.69 |
| Specimen | Serum 1 | 4.52 | 3.70 | −18.14 | 3.00 | −33.63 |
| | Serum 2 | 5.31 | 5.20 | −2.07 | 4.37 | −17.70 |
| | Serum 3 | 3.26 | 4.00 | 22.70 | 3.37 | 3.37 |
| | Serum 4 | 5.48 | 7.30 | 33.21 | 6.30 | 14.96 |
| | Serum 5 | 4.37 | 5.50 | 25.86 | 6.51 | 48.97 |
| | Serum 6 | 4.94 | 4.90 | −0.81 | 5.60 | 13.36 |
| | Serum 7 | 7.11 | 8.40 | 18.14 | 6.60 | −7.17 |
| | Serum 8 | 4.42 | 4.70 | 6.33 | 4.30 | −2.71 |
| | Serum 9 | 4.28 | 6.71 | 56.78 | 6.70 | 56.54 |
| | Serum 10 | 4.46 | 4.20 | −5.83 | 4.00 | −10.31 |
| | Serum 11 | 42.58 | 48.45 | 13.79 | 44.60 | 4.74 |
| | Serum 12 | 16.57 | 21.10 | 27.34 | 13.30 | −19.73 |
| | Serum 13 | 74.70 | 78.20 | 4.69 | 75.50 | 1.07 |
| | Serum 14 | 21.07 | 25.70 | 21.97 | 22.20 | 5.36 |
| | Serum 15 | 50.43 | 53.60 | 6.29 | 55.34 | 9.74 |
| | Serum 16 | 40.76 | 45.00 | 10.40 | 43.60 | 6.97 |
| | Serum 17 | 24.73 | 32.00 | 29.40 | 28.10 | 13.63 |
| | Serum 18 | 29.00 | 39.46 | 36.07 | 32.20 | 11.03 |
| | Serum 19 | 57.97 | 60.30 | 4.02 | 54.20 | −6.50 |

### Reference Interval Determination

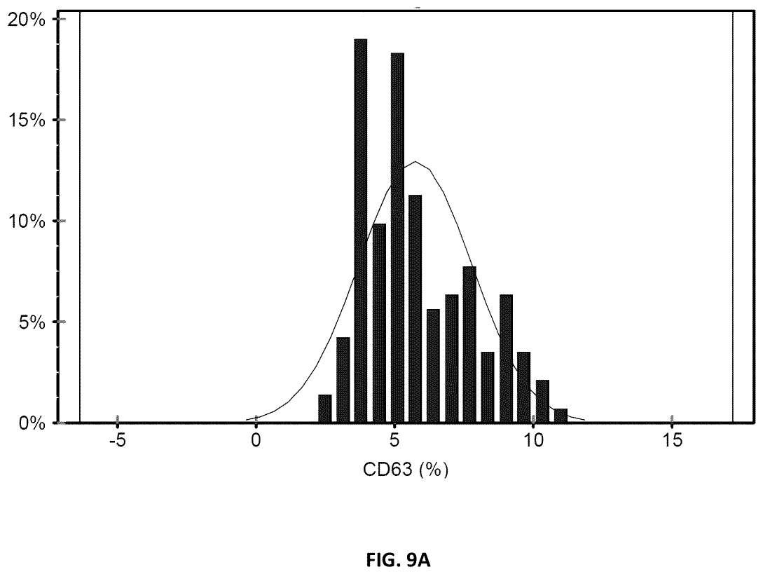

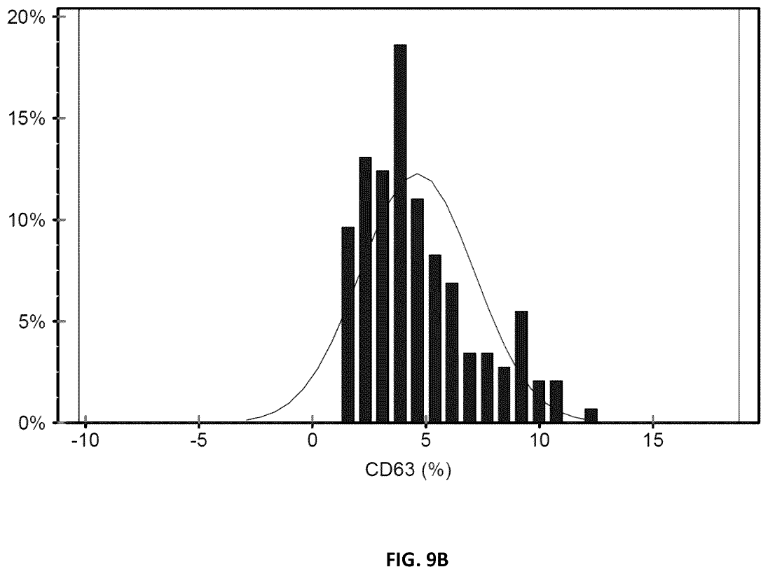

The upper limit of the reference interval determined from a population of 142 individuals with total IgE<80 IU/L, was determined to be 10.61% (9.79 to 11.49, 95% CI) (FIG. 9A). This threshold was verified in a separate analysis of 145 samples negative BHRA results <2.2 CU IndexR (post Chauvenet outlier exclusion) where the upper limit of the reference interval was determined to be 11.52% (10.19 to 13.02, 95% CI) (FIG. 9B).

Thus, FIG. 9A shows determination of the reference interval. A total of 144 low IgE (<80 IU/mL) serum were screened via PD-BAT, 2 specimens were excluded by the Chauvenet method. FIG. 9A shows the distribution of the 142 low IgE sera used to acquire the reference interval. Transformed parametric analysis via EP Evaluator provides a reference interval of 10.61% activated basophils. Mean, 5.79% CD63+; standard deviation, 1.98; median, 5.34; range, 2.15 to 11.24. FIG. 9B shows verification of the reference interval. A total of 157 strong negative (<2.2 on CU IndexR) BHRA serum specimens were screened via PD-BAT, 12 specimens were excluded by the Chauvenet method. FIG. 9B shows the distribution of the 145 sera used to verify the reference interval. Transformed parametric analysis via EP Evaluator provides a reference interval of 11.52% activated basophils. Mean, 4.71%; standard deviation, 2.47; median, 4.0; range, 1.3 to 11.9. The establishment of a robust reference interval provides a crucial foundation of the application of PD-BAT.

### Basophil Histamine Release Assay Comparison to PD-BAT

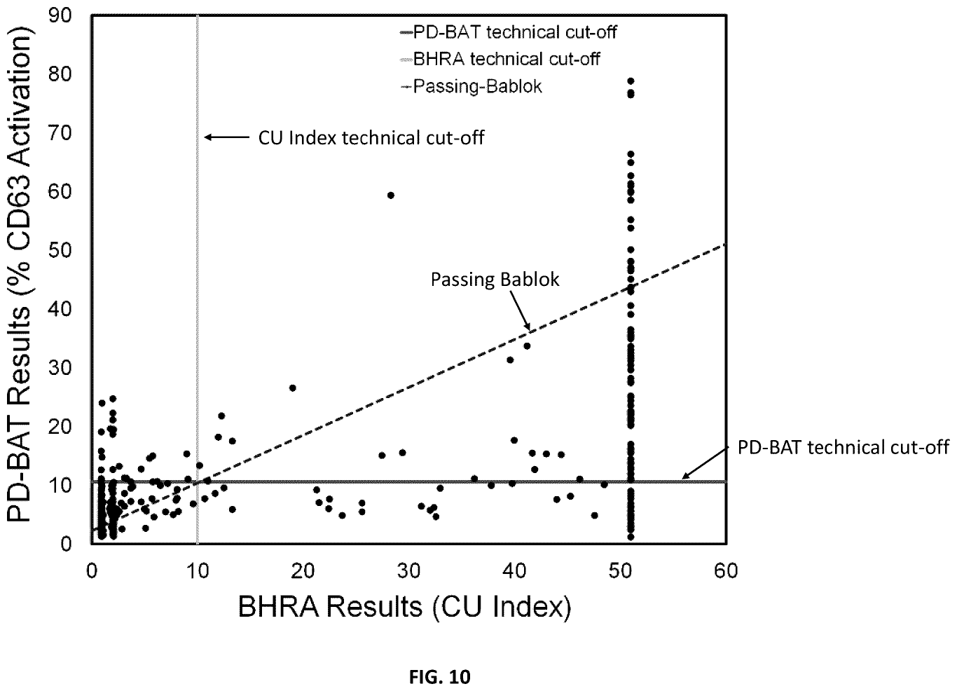

Comparison of result obtained for 343 samples tested by the CU-Index and PD-BAT revealed an overall percent agreement of 79.9% (FIG. 10, Table 6). In these experiments, serum spanning the BHRA analytical spectrum were screened with PD-BAT, results were analyzed via EP Evaluator. Shown is the PD-BAT technical cut-off (10.61) (horizontal red line) and the CU IndexR technical cut-off (>10 CU Index Units) (vertical yellow line). The Passing Bablok linear regression slope of 0.814 (dotted line) accounting for the proportional and systematic differences between the two methods, suggests a moderate linear relationship between the BHRA and PD-BAT. 28% of the patients tested had strong positive results for the CU Index (>50 CU IndexR Units). Of these strong positive samples, 75 sera (78.1%) were positive by the PD-BAT. In summary the method comparison between the donor pooling method for BAT and BHRA, spanning various sample values, indicates substantial agreement. This emphasizes that the PD-BAT method produces similar results to established assays.

TABLE 6
Quantitative comparison of CU Index ® vs. PD-BAT

| | BHRA Specimens |
|---|---|
| Statistical Analysis | (n = 343) |
| Agreement (%) | 79.9 |
| Sensitivity (%) | 78.1 |
| Specificity (%) | 80.6 |
| Positive Agreement (%) | 78.1 |
| Negative Agreement (%) | 80.6 |
| Prevalence (%) | 28.0 |
| Predictive Value Positive (%) | 61.0 |
| Predictive Value Negative (%) | 90.5 |

### Method Comparison to Individual Donor CSU BAT

A critical aspect of the study focused on comparing the Pooled Donor Basophil Activation Test (PD-BAT) with the conventional method using individual donors for Chronic Spontaneous Urticaria Basophil Activation Test (CSU-BAT). Nine healthy blood donors were prescreened for use in CSU-BAT (using individual donors). Three of these were selected based on a high CD63+ response to anti-FcεRI (77.3%, 77.5%, and 93.0%, respectively) and a low response to a normal serum pool (3.6%, 2.8%, 6.6% CD63+ respectively). CSU-BAT was performed with 69 sera from patients suspected of having CSU with 3 donors and the results are compared to the PD-BAT (average of results from two runs) (FIGS. 11A, 11B and 11C showing results for donors 1, 2 and 3, respectively). The correlation coefficients showed variations among donors. Notably, donors 1, and 2 met the PD-BAT criteria for inclusion in the final pool, displaying moderate correlation of 0.85 and 0.92 with the PD-BAT results. However, donor 3 had a lower correlation of 0.59 with the average PD-BAT results.

For analysis of method agreement, the average of duplicate PD-BAT tests (on the x-axis) is regarded as the “true” value. Table 7 shows that positive predictive value as well as positive and negative percent agreement strongly depend on the individual basophil donor. For Table 7, TP: true positives, TN: true negatives, FP: false positives, FN: false negatives, PPA: positive percent agreement, NPA: negative percent agreement, PPV: positive predictive value, NPV: negative predictive value.

TABLE 7
Method comparison of individual donor results to pooled PD-BAT

| Basophil | | | | | | | | |
|---|---|---|---|---|---|---|---|---|
| Donor # | TP | TN | FP | FN | PPA | NPA | PPV | NPV |
| 1 | 4 | 60 | 2 | 3 | 57.1% | 96.8% | 66.7% | 95.2% |
| 2 | 6 | 60 | 2 | 1 | 85.7% | 96.8% | 75.0% | 98.4% |
| 3 | 5 | 43 | 19 | 2 | 71.4% | 69.4% | 20.8% | 95.6% |

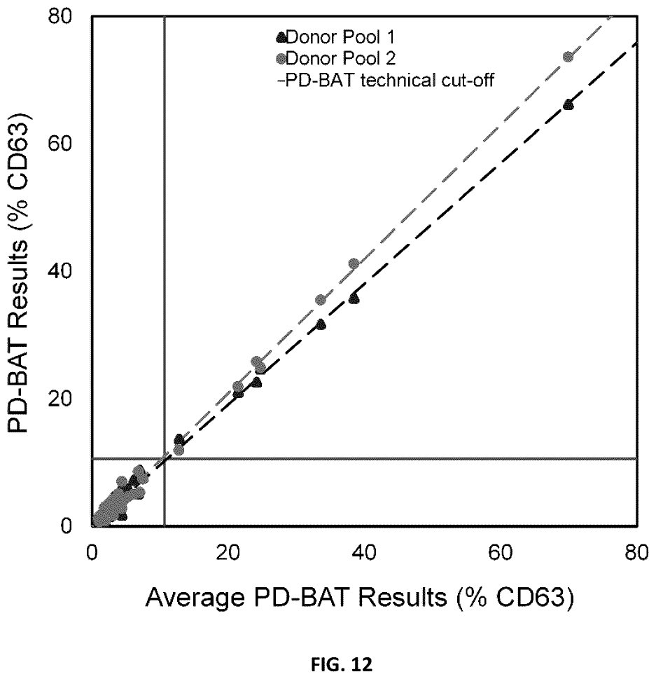

The 69 samples were tested twice by the PD-BAT using two separate basophil donor pools, showing consistency between runs (FIG. 12).

### Discussion

The application of basophil activation testing for the assessment of patients with CSU by both flow cytometry and histamine release approaches is challenging due the intrinsic variability of individual donors. This variability has been observed in prior screening processes; it was found that a subset of screened donors performed sub-optimally in the assay of characterized controls and patient serum pools (FIGS. 5A and 5B). To address this, some laboratories test patient serum samples with multiple donors and use arbitrary criteria to judge the positivity of patient results (Gentinetta et al., 2011, Marcelino et al., 2021; D'Auria et al., 2019). It was found, however, that results obtained using individual donors, even with prescreening to eliminate non-responders and donors that produced elevated background activation, were not consistent for many of the patients tested (FIGS. 11A, 11B and 11C, Table 7). By pooling multiple donors that met defined performance specifications, it was possible to achieve more consistent batch to batch performance of a flow-cytometry based assay (FIG. 12).

Along with improving assay performance, the donor pooling approach served to improve instrument and reagent use efficiency. Flow cytometer use time was reduced by pre-selecting donors with relatively high basophil counts which served to reduce the time needed to count the required number of basophils relative to individual donors with lower basophil counts. The pre-screening and pooling approach requires more labor and reagent when used for small batch testing. Prescreening 30 candidate donors requires that 90 individual tests be performed (for background, LSP and anti-FcεRI controls). However, for actual patient testing only one donor pool needs to be tested compared to at least three individual donors tested by prior methods (Gentinetta et al., 2011, Marcelino et al., 2021; D'Auria et al., 2019).

An intriguing observation within this study pertains to changes in the stability of pooled whole blood, particularly two hours after the pooling process, which may be attributed to a mixed lymphocyte reaction (MLR). MLR is a phenomenon in which T lymphocytes from one individual react with the major histocompatibility complex (MHC) antigens of another individual. In the context of this investigation, the mixed lymphocyte reaction within the donor blood pool could contribute to the precipitous decline in activation levels, especially as observed for the high serum pool. It was found that sample testing should be initiated within two hours of pool formation for optimal results. However, once the samples and donor mixing steps are complete and the samples are processed (stimulated, stained, lysed and washed), the actual sample acquisition can be delayed for up to three days.

The moderate correlation observed between the PD-BAT and the CU-Index (FIG. 10) is consistent with results of other studies where histamine release- and flow cytometric activation marker-based methods have been compared (Schoepke et al. 2019). The data in this example revealed an excellent inter-assay comparability of the results produced by the PD-BAT (FIGS. 6 and 12), ostensibly due to the consistent performance of multiple donor pools. It is possible that the lack of direct comparability of results might, in part, be related to inconsistent performance of individual donors employed in the CU-IndexR as we observed for individual donors in the CSU-BAT, specifically with Donor 3 (FIG. 11C). Donor 3 would not have been chosen for the PD-BAT assay since it does not meet the criteria required for inclusion in the pool. Not surprisingly, this donor also had a poor correlation with the average PD-BAT result. These findings underscore the PD-BAT's ability to minimize donor-related variations, ensuring reliable and consistent results.

### Example 4—Embodiments

The disclosure may be better understood by references the following non-limiting embodiments.

A.1 A method to pool biological samples for use in testing comprising: screening a plurality of biological samples for a response indicative of the presence of a predetermined indication; and selecting a plurality of the samples that provide the response above a first predetermined threshold to generate the pool.

A.2 The method of any of the previous or subsequent method embodiments, further comprising: screening the plurality of samples to determine whether the samples have a non-specific response above a different predetermined threshold; and removing samples which have a non-specific response from the pool.

A.3 The method of any of the previous or subsequent method embodiments, wherein the biological samples are blood.

A.4 The method of any of the previous or subsequent method embodiments, wherein the pool comprises at least 10, or at least 20, or at least 30 biological samples.

A.5 The method of any of the previous or subsequent method embodiments, wherein the response is basophil activation.

A.6 The method of any of the previous or subsequent method embodiments, wherein the basophil activation is assessed using anti-FcεRI mAb activation of CD63 as a marker of basophil degranulation.

A.7 The method of any of the previous or subsequent method embodiments, wherein the predetermined indication is chronic spontaneous urticaria.

A.8 The method of any of the previous or subsequent method embodiments, further comprising evaluating the precision of the pool by comparing results to: (i) a first pool of additional samples that generate a high response at or above a first predetermined quality control (QC) threshold; (ii) a second pool of additional samples that generate a medium response at a second predetermined QC threshold; and (iii) a third pool of additional biological samples that generate a low response at a third predetermined QC threshold.

A.9 The method of any of the previous or subsequent method embodiments, wherein the additional biological samples are serum.

A.10 The method of any of the previous or subsequent method embodiments, wherein the technical cutoff for the pooled samples as indicating a positive response indicative of the presence of a predetermined indication is defined using a 95% confidence index (CI) of a plurality of normal samples.

A.11 A method to test for a response indicative of the presence of a predetermined indication comprising in a sample from a subject comprising the steps of: generating a plurality of pooled samples that provide the response above a first predetermined threshold and optionally, do not exhibit a non-specific response; and adding a portion of the sample from the subject to the pooled samples of (a) to determine if the sample generates the response.

A.12 The method of any of the previous or subsequent method embodiments, wherein the pooled samples are blood.

A. 13 The method of any of the previous or subsequent method embodiments, wherein the pooled samples comprise at least 10, or at least 20, or at least 30 biological samples.

A. 14 The method of any of the previous or subsequent method embodiments, wherein the response is basophil activation.

A.15 The method of any of the previous or subsequent method embodiments, wherein the basophil activation is assessed using anti-FcεRI mAb activation of CD63 as a marker of basophil degranulation.

A.16 The method of any of the previous or subsequent method embodiments, wherein the predetermined indication is chronic spontaneous urticaria.

A.17 The method of any of the previous or subsequent method embodiments, wherein the sample from the subject is serum.

A.18 The method of any of the previous or subsequent method embodiments, further comprising evaluating the precision of the pool by comparing results to: (i) a first pool of additional serum samples that generate a high response at or above a first predetermined quality control (QC) threshold; (ii) a second pool of additional serum samples that generate a medium response at a second predetermined QC threshold; and (iii) a third pool of additional serum samples that generate a low response at a third predetermined QC threshold.

A.19 The method of any of the previous or subsequent method embodiments, wherein the technical cutoff for the pooled samples as indicating a positive response indicative of the presence of a predetermined indication is defined using a 95% CI of a plurality of normal samples.

B.1 A composition or kit for performing any of the disclosed method embodiments or using any of the disclosed system or computer program embodiments.

B.2 A composition or kit comprising a pool of biological samples for screening a sample from a subject for a predetermined indication, wherein the pool comprises a plurality biological samples each exhibiting a response above a first predetermined threshold and optionally do not exhibit a non-specific response, and wherein the response is indicative of the presence of the predetermined indication and optionally comprising instructions for use.

B.3 The composition or kit of any of the previous or subsequent composition or kit embodiments, wherein the predetermined indication is an auto-antibody disease.

B.4 The composition or kit of any of the previous or subsequent composition or kit embodiments, wherein the disease is chronic spontaneous urticaria (CSU).

B.5 The composition or kit of any of the previous or subsequent composition or kit embodiments, wherein the biological samples in the pool are whole blood samples.

B.6 The composition or kit of any of the previous or subsequent composition or kit embodiments, wherein the pool comprises at least 10, at least 20, or at least 30 biological samples.

B.7 The composition or kit of any of the previous or subsequent composition or kit embodiments, wherein the response is basophil activation.

B.8 The composition or kit of any of the previous or subsequent composition or kit embodiments, wherein the basophil activation is assessed using anti-FcεRI mAb activation of CD63 as a marker of basophil degranulation.

C.1 A system for performing any of the previously disclosed method embodiments or for using any of the previously disclosed composition or kit embodiments.

C.2 A system for generating a pool of biological samples for use in testing comprising: a component or station for screening a plurality of biological samples for a response indicative of the presence of a predetermined indication; and a component or station for selecting a plurality of the samples that provide the response above a first predetermined threshold to generate the pool.

C.3 The system of any of the previous or subsequent system embodiments, further comprising a component or station for assessing a biological sample from a subject for the response, the biological sample from the subject being distinct from any of the biological samples used to generate the pool, to determine if the sample generates the response and to quantify the level of the response as compared to the pools.

C.4 The system of any of the previous or subsequent system embodiments, further comprising a component or station for screening the plurality of biological samples to determine whether the samples have a non-specific response; and removing the biological samples which have a non-specific response from inclusion in the pool.

C.5 The system of any of the previous or subsequent system embodiments, wherein the biological samples screened for inclusion in the pool are blood.

C.6 The system of any of the previous or subsequent system embodiments, wherein the pool comprises at least 10, or at least 20, or at least 30 biological samples.

C.7 The system of any of the previous or subsequent system embodiments, wherein the response is basophil activation.

C.8 The system of any of the previous or subsequent system embodiments, wherein the basophil activation is assessed using anti-FcεRI mAb activation of CD63 as a marker of basophil degranulation.

C.9 The system of any of the previous or subsequent system embodiments, wherein the predetermined indication is chronic spontaneous urticaria.

C.10 The system of any of the previous or subsequent system embodiments, further comprising a station and/or component for evaluating the precision of the pool.

C.11 The system of any of the previous or subsequent system embodiments, wherein the station and/or component for evaluating the precision of the pool comprises a station or component for comparing results to: (i) a first pool of additional biological samples that generate a high response at or above a first predetermined quality control (QC) threshold; (ii) a second pool of additional biological samples that generate a medium response at a second predetermined QC threshold; and (iii) a third pool of additional biological samples that generate a low response at a third predetermined QC threshold.

C.12 The system of any of the previous or subsequent system embodiments, wherein the additional biological samples are serum.

C.13 The system of any of the previous or subsequent system embodiments, wherein a technical cutoff for the pooled samples as indicating a positive response indicative of the presence of a predetermined indication is defined using a 95% CI of a plurality of normal samples.

C.14 The system of any of the previous or subsequent system embodiments, further comprising a computer-program product tangibly embodied in a non-transitory machine-readable storage medium including instructions configured to run any of the components or stations of the system.

D.1 A computer-program product tangibly embodied in a non-transitory machine-readable storage medium including instructions configured to perform any of the methods of the previous method embodiments, or use any of the compositions of the previous composition embodiments, or run any of the stations and/or components of any of the systems embodiments.

D.2 A computer-program product tangibly embodied in a non-transitory machine-readable storage medium including instructions configured to: screen a plurality of biological samples for a response indicative of the presence of a predetermined indication; and select a plurality of the samples that provide the response above a first predetermined threshold to generate the pool.

D.3 A computer-program product tangibly embodied in a non-transitory machine-readable storage medium including instructions configured to: generate a plurality of pooled samples that provide the response above a first predetermined threshold and optionally, do not exhibit a non-specific response; and add a portion of the sample from the subject to the pooled samples to determine if the sample generates the response.

D.4 A computer-program product tangibly embodied in a non-transitory machine-readable storage medium including instructions configured to run a system comprising: a component or station for screening a plurality of biological samples for a response indicative of the presence of a predetermined indication; and a component or station for selecting a plurality of the samples that provide the response above a first predetermined threshold to generate the pool.

D.5 The computer program product of any of the previous or subsequent computer program embodiments, further comprising instructions configured to run a system comprising a component or station to use the pool to test for a response indicative of the presence of the predetermined indication in a sample from a subject.

That which is claimed is:

1. A method to pool biological samples for use in testing comprising the steps of:
(a) screening a plurality of biological samples for a response indicative of the presence of a predetermined indication; and
(b) selecting a plurality of the samples that provide the response above a first predetermined threshold to generate the pool.

2. The method of claim 1, further comprising the steps of:
(a) screening the plurality of samples to determine whether the samples have a non-specific response above a different predetermined threshold; and
(b) removing samples which have a non-specific response from the pool.

3. The method of claim 1, wherein the biological samples are blood.

4. The method of claim 1, wherein the pool comprises at least 10, or at least 20, or at least 30 biological samples.

5. The method of claim 1, wherein the response is basophil activation.

6. The method of claim 5, wherein the basophil activation is assessed using anti-FcεRI mAb activation of CD63 as a marker of basophil degranulation.

7. The method of claim 6, wherein the predetermined indication is chronic spontaneous urticaria.

8. The method of claim 1, further comprising evaluating the precision of the pool by comparing results to: (i) a first pool of additional samples that generate a high response at or above a first predetermined quality control (QC) threshold; (ii) a second pool of additional samples that generate a medium response at a second predetermined QC threshold; and (iii) a third pool of additional biological samples that generate a low response at a third predetermined (QC) threshold.

9. The method of claim 8, wherein the additional biological samples are serum.

10. The method of claim 1, wherein a technical cutoff for the pooled samples as indicating a positive response indicative of the presence of a predetermined indication is defined using a 95% CI of a plurality of normal samples.

11. A method to test for a response indicative of the presence of a predetermined indication comprising in a sample from a subject comprising the steps of:
(a) generating a plurality of pooled samples that provide the response above a first predetermined threshold and optionally, do not exhibit a non-specific response; and
(b) adding a portion of the sample from the subject to the pooled samples of (a) to determine if the sample generates the response.

12. The method of claim 11, wherein the pooled samples are blood.

13. The method of claim 11, wherein the pooled samples comprise at least 10, or at least 20, or at least 30 biological samples.

14. The method of claim 11, wherein the response is basophil activation.

15. The method of claim 14, wherein the basophil activation is assessed using anti-FcεRI mAb activation of CD63 as a marker of basophil degranulation.

16. The method of claim 14, wherein the predetermined indication is chronic spontaneous urticaria.

17. The method of claim 11, wherein the sample from the subject is serum.

18. The method of claim 17, further comprising evaluating the precision of the pool by comparing results to: (i) a first pool of additional serum samples that generate a high response at or above a first predetermined quality control (QC) threshold; (ii) a second pool of additional serum samples that generate a medium response at a second predetermined QC threshold; and (iii) a third pool of additional serum samples that generate a low response at a third predetermined QC threshold.

19. The method of claim 11, wherein a technical cutoff for the pooled samples as indicating a positive response indicative of the presence of a predetermined indication is defined using a 95% CI of a plurality of normal samples.

20. A composition comprising a pool of biological samples for screening a sample from a subject for a predetermined indication, wherein the pool comprises a plurality of biological samples each exhibiting a response above a first predetermined threshold and optionally do not exhibit a non-specific response, and wherein the response is indicative of the presence of the predetermined indication.

21. A system for generating a pool of biological samples for use in testing comprising:
(a) a component or station for screening a plurality of biological samples for a response indicative of the presence of a predetermined indication; and
(b) a component or station for selecting a plurality of the samples that provide the response above a first predetermined threshold to generate the pool.
